(19) United States
(12) Patent Application Publication
(10) Pub. No.: US 2024/0115494 A1
(43) Pub. Date: Apr. 11, 2024

(54) ENCAPSULATED COMPOSITIONS AND METHOD OF USE - AFFECTING SATIETY

(71) Applicant: Nutragenom, LLC, Las Vegas, NV (US)

(72) Inventor: Eugene P. Pittz, Las Vegas, NV (US)

(21) Appl. No.: 18/263,905

(22) PCT Filed: Feb. 2, 2022
(86) PCT No.: PCT/US2022/014963

§ 371 Date: Aug. 2, 2023

Related U.S. Application Data
(60) Provisional application No. 63/206,171, filed on Feb. 3, 2021.

(51) Int. Cl. A61K 9/00; A61K 9/48; A61K 31/723; A61K 31/736; A61K 33/06; A61P 3/04

(52) U.S. Cl. CPC A61K 9/0065; A61K 9/4866; A61K 31/723; A61K 31/736; A61K 33/06; A61P 3/04

(57) ABSTRACT

A composition and method is described herein. The method includes administrating a composition capable of generating molecular hydrogen to an individual, the method comprising: co-administering food and a dose or doses of the composition to the alimentary canal of the individual, wherein the composition comprises glucomannan, xanthan gum, magnesium metal powder, an organic acid, excipients, or any combination thereof. The method also includes administering the composition formulated for oral delivery wherein the administration induces satiety/weight loss, exercise endurance, anti-inflammatory effects, or any combination thereof. The method also includes administering the composition enclosed in a capsule, as a powder composition, as a hydrogel, or any combination thereof.

### CROSS-REFERENCE

This application claims the benefit of U.S. Patent Application No. 63/206,171 filed Feb. 3, 2021, which is hereby incorporated by reference in its entirety.

### BACKGROUND

Current methods of administrating molecular hydrogen (H2 gas) for wellness, anti-aging, weight management, treatment or prevention of various inflammatory diseases or metabolic syndromes in humans and animals are limited in their capacity to sustain controlled administration of molecular hydrogen. The health benefits of administering molecular hydrogen to humans and animals have been identified as ranging from attenuating oxidative stress, improving cellular function, reducing inflammation, improving redox regulation, and improving cellular homeostasis. Overall, the potential of administering molecular hydrogen to alleviate the severity or prevalence of diseases related to oxidative stress has been demonstrated.

Molecular hydrogen is readily absorbed into tissues. The duration of H2 in the body is short-lived, since it readily diffuses in and out of tissues. Sustaining molecular hydrogen in the tissues of humans and animals is needed for increasing the efficacy of molecular hydrogen for reduction of tissue damage, effects on slowing aging and slowing the progression of chronic degenerative diseases as well as for body weight control. However, standard practices for administration of molecular hydrogen by various routes, including IV, oral, transdermal, and inhalation limit molecular hydrogen dosage, and thus the potential benefits of administration. A means of generating and retaining molecular hydrogen in the body, in a safe, economic, and consumer-friendly manner, is needed to advance its use as a therapeutic agent.

Alternatives to known approaches could range from introducing new components to the formulation comprising molecular hydrogen to adjusting methods of administration to ensure sustained release, thereby increasing efficiency.

### SUMMARY

The present invention and its embodiments relate to methods of administrating a composition capable of generating molecular hydrogen to an individual attenuate oxidative stress, induce satiety, weight loss, exercise endurance, anti-inflammatory effects, or any combination thereof. In particular, the present invention describes a composition and a method. The composition comprises glucomannan, xanthan gum, magnesium metal powder, an organic acid, excipients, or any combination thereof. The method comprises co-administering food and a dose or doses of the composition to the alimentary canal of the individual, wherein the composition is a hydrogel formulation, a powder formulation, or enclosed in a capsule. The method includes numerous process steps, such as: contacting the powder formulation with water to form a hydrogel prior to co-administration of the hydrogel and food. It should be appreciated that in some embodiments, the method also includes contacting the powder formulation with acidic solution in individual's stomach acid following co-administration of food and the capsule or capsules comprising the powder formulation. In some embodiments, the present invention also includes expanding the powder composition in water to form a hydrogel prior to co-administering with food to the individual. In some embodiments, the present invention also includes expanding the powder composition in water to form a hydrogel prior to co-administering with food and whith a capsule or capsules comprising the powder formulation to the individual. In some embodiments, the present invention also comprises encapsulated and powder compositions that form expansive hydrogels, affecting satiety when the capsules and/or hydrogels are ingested. The resultant hydrogels can occupy up to about 15% to about 60% of the individual's stomach capacity for at least about 4 hours to at least about 7 hours and their methods of administration. The ingested expansive hydrogels thereby affecting satiety, which can induce satiety, weight loss, exercise endurance, anti-inflammatory effects, or any combination thereof.

Described herein in one aspect, is a method of administrating a composition capable of generating molecular hydrogen to an individual, the method comprising: co-administering food and a dose or doses of the composition to the alimentary canal of the individual, wherein the composition comprises glucomannan, xanthan gum, magnesium metal powder, or any combination thereof. In some embodiments, the composition is enclosed in a capsule. In some embodiments, the composition is formulated for oral delivery. In some embodiments, the composition is formulated to induce satiety/weight loss. In some embodiments, the composition is formulated to induce exercise endurance. In some embodiments, the composition is formulated to induce anti-inflammatory effects. In some embodiments, co-administration of food and a dose of the composition comprises releasing the composition into the stomach of the individual at most about 40 minutes following the introduction of food into the stomach of the individual. In some embodiments, composition comprises at least about 2 wt % to at least about 98 wt % of glucomannan. In some embodiments, the composition comprises from about 40 wt % to about 50 wt % of glucomannan. In some embodiments, the composition comprises at least about 0.0 wt % to at least about 98 wt % of xanthan gum. In some embodiments, the composition comprises from about 40 wt % to about 50 wt % of xanthan gum. In some embodiments, the composition comprises at least about 0.001 wt % to at least about 30 wt % of magnesium metal powder. In some embodiments, the composition comprises from about 0.001 wt % to about 5 wt % of magnesium metal powder. In some embodiments, the composition is released from the capsule following co-administration. In some embodiments, the composition is enclosed in the capsule that is at least 0, 00, or 000 in size. In some embodiments, the capsule comprises cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate, hydroxypropyl methylcellulose (HPMC), gelatin, polysaccharide, or any combination thereof. In some embodiments, the capsule is made from hydroxypropyl methylcellulose (HPMC), cellulose, gelatin, or any combination thereof. In some embodiments, the method comprises co-administration of food and at least 2 capsules comprising a dose of the composition. In some embodiments, the method comprises co-administration of food and at least 3 capsules comprising a dose of the composition. In some embodiments, following co-administration of food and at least one capsule comprising a dose of the composition, the composition expands to a volume of about 100 milliliters to about 2,000 milliliters in acidic solution to form a hydrogel. In some embodiments, the composition expands to a volume of about 300 milliliters. In some embodiments, following co-administration of food and at least one capsule comprising a dose of the composition, the composition associates with acidic solution of the individual's stomach to form a hydrogel that expands to a volume of about 100 times to about 200 times the original volume of the capsule. In some embodiments, the composition associates with acidic solution of the individual's stomach to form a hydrogel that expands to a volume of about 300 times the original volume of the capsule. In some embodiments, following co-administration of food and at least one capsule comprising a dose of the composition, the hydrogel comprises a volume equivalent to about 15% to about 60% of the individual's stomach capacity. In some embodiments, the hydrogel comprises a volume sufficient to induce satiety in the individual. In some embodiments, the hydrogel comprises a volume sufficient to induce weight loss in the individual. In some embodiments, the hydrogel remains at a constant volume within the stomach of the individual for at least about 4 hours to at least about 7 hours. In some embodiments, the composition expands in acidic solution to form a hydrogel, generating molecular hydrogen by reaction of magnesium metal powder with the acidic solution. In some embodiments, the molecular hydrogen accelerates the dissolution of the capsule containing the composition in the acidic solution. In some embodiments, the molecular hydrogen induces satiety/weight loss, exercise endurance, anti-inflammatory effects, or any combination thereof. In some embodiments, the acidic solution is stomach acid. In some embodiments, satiety or weight loss is induced following co-administration of food and at least one capsule comprising a dose of the composition when ingested at least once a day. In some embodiments, daily co-administration of food and at least one capsule comprising a dose of the composition reduces body weight, induces exercise endurance, and/or induces anti-inflammatory effects in the individual over an extended period of time. In some embodiments, an extended period of time comprises at least about 1 week. In some embodiments, an extended period of time comprises about 1 month to about 50 years. In some embodiments, the capsules can be administered at least once a day. In some embodiments, a dose of the composition, is about 0.80 grams to about 1.50 grams. In some embodiments, the individual is a human individual. Described herein in one aspect, is a powder composition for use in an individual, wherein the powder composition comprises glucomannan, xanthan gum, magnesium metal powder, an organic acid, excipients, or any combination thereof. In some embodiments, the powder composition is (i) contacted with water to form a hydrogel, and (ii) co-administered with food into the alimentary canal of an individual. In some embodiments, the powder composition for use in inducing satiety/weight loss in an individual. In some embodiments, the powder composition for use in inducing exercise endurance in an individual. In some embodiments, the powder composition for use in inducing anti-inflammatory effects in an individual. In some embodiments, a single dose of the powder composition comprises from about 3.0 grams to about 10.0 grams of glucomannan (GMN), xanthan gum (XG), magnesium metal powder (MMP), or any combination thereof. In some embodiments, the powder composition further comprises from about 30 wt % to about 60 wt % of GMN. In some embodiments, the powder composition further comprises from about 30 wt % to about 60 wt % of XG. In some embodiments, the powder composition further comprises from about 0.1 wt % to about 20 wt % of MMP. In some embodiments, the powder composition further comprises from about 0.01 wt % to about 25 wt % of an organic acid. In some embodiments, the powder composition further comprises from about 0.001 wt % to about 10 wt % of an excipient. In some embodiments, the composition comprises an organic acid such as citric acid, malic acid, succinic acid, tartaric acid, adipic acid, lactic acid, or any combination thereof. In some embodiments, the composition comprises excipients such as sweeteners, antioxidants, anticaking agents, flavoring agents, coloring agents, or any combination thereof. In some embodiments, the composition comprises sweeteners such as sucralose, stevia, sugar alcohol (e.g., erythritol), acesulfame, sucrose, glucose, fructose, aspartame, saccharin, cyclamate, agarose, or any combination thereof. In some embodiments, the composition comprises antioxidants such as ascorbic acid, isoascorbic acid, vitamin E, polyphenols, or any combination thereof. In some embodiments, the composition comprises anticaking agents such as tricalcium phosphate, powdered cellulose, magnesium stearate, sodium bicarbonate, sodium silicate, stearic acid, or any combination thereof. In some embodiments, the composition comprises flavoring agents such as lemon, chocolate, cherry, banana, pineapple, grape, wintergreen, or any combination thereof. In some embodiments, the composition comprises coloring agents such as riboflavin, carmel, annatto, chlorella, turmeric, elderberry, or any combination thereof. In some embodiments, following co-administration, the hydrogel expands by a volume of about 80 times to about 200 times the volume originally occupied by the powder composition contacted with water. In some embodiments, prior to co-administration, the powder composition expands to form a hydrogel, comprising a volume equivalent to about 15% to about 60% of the individual's stomach capacity. In some embodiments, up to four doses of the powder composition in hydrogel form comprises a volume of about 8 ounces to about 16 ounces. In some embodiments, the hydrogel comprises a viscosity of at least about 15,000 millipascal-second at a temperature of about 72 degrees Fahrenheit. In some embodiments, the hydrogel comprises a volume sufficient to induce satiety in the individual. In some embodiments, the hydrogel comprises a volume sufficient to induce weight loss in the individual. In some embodiments, the hydrogel remains at a constant volume within the stomach of the individual for at least about 3 hours to at least about 8 hours. In some embodiments, the powder composition is packaged as a powder. In some embodiments, the powder composition is packaged in about 30 doses to about 60 dose packets. In some embodiments, the powder composition is packaged in about 5 grams to about 10 grams single dose packets. In some embodiments, the powder composition is enclosed in a capsule to form encapsulated composition. In some embodiments, the encapsulated composition is enclosed in the capsule that is at least 0, 00, or 000 in size. In some embodiments, the encapsulated composition is enclosed in the capsule comprising cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate, hydroxypropyl methylcellulose (HPMC), gelatin, polysaccharide, or any combination thereof. In some embodiments, the encapsulated composition is enclosed in the capsule comprising hydroxypropyl methylcellulose (HPMC), cellulose, gelatin, or any combination. In some embodiments, the encapsulated composition is released from the capsule following co-administration. In some embodiments, the powder composition and/or hydrogel can be co-administered at least once a day. In some embodiments, the powder composition, encapsulated composition, and/or hydrogel can be co-administered at least once a day.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an image of three size 000 capsules containing 1:1 glucomannan:xanthan gum dissolved in 300 mL of 5% acetic acid for 120-minutes in a Griffin beaker flask with flat bottom, according to at least some embodiments disclosed herein.

FIG. 2 depicts an image of the top view of the capsular contents of the same preparation depicted in FIG. 1, according to at least some embodiments disclosed herein.

FIG. 3 depicts an image of four capsules containing glucomannan (NOW® Foods) in aqueous-acidic solution, according to at least some embodiments disclosed herein.

FIG. 4 depicts an image of (A) three capsules containing 1T (see Table 3) dissolved in 350 mL of a vinegar solution in a cup, (B) 99.7 grams of a simulated food sample dissolved in 650 mL of 5% acetic acid in a cup, according to at least some embodiments disclosed herein.

FIG. 5 depicts an image of the contents of the same preparations depicted in FIGS. 4A and 4B in a Griffin beaker flask prior to mixing, according to at least some embodiments disclosed herein.

FIG. 6 depicts an image of the contents of the same preparations depicted in FIGS. 4A and 4B in a Griffin beaker flask after mixing, according to at least some embodiments disclosed herein.

FIG. 7 depicts a graph of molecular hydrogen measured in breath of individual (as parts per million) after co-administration of food and three capsules containing 1T as a function of time (in minutes), according to at least some embodiments disclosed herein.

FIG. 8 depicts an image of (A) three size 000 capsules containing 1:1.2 glucomannan:xanthan gum+0.667 grams of magnesium metal powder (MMP) (Capsule 1U; see Table 4) dissolved in 100 mL of 5% acetic acid 60 minutes after mixing in a Griffin beaker flask with flat bottom, (B) three size 000 capsules containing 1:1.2 glucomannan:xanthan gum+1.043 grams of magnesium metal powder (Capsule 4U; see Table 4) dissolved in 100 mL of 5% acetic acid 60 minutes after mixing in a Griffin beaker flask with flat bottom, according to at least some embodiments disclosed herein.

FIG. 9 depicts an image of the same preparations depicted in FIG. 8, about 6 hours post-mixing, according to at least some embodiments disclosed herein.

FIG. 10 depicts an image of a hydrogel created after mixing two size 000 capsules of 1U (Table 4) with 400 mL of vinegar solution after addition of another 400 mL of vinegar solution in a Griffin beaker flask with flat bottom, according to at least some embodiments disclosed herein.

FIG. 11 depicts a graph of viscosity for three GMP/XG/MMP dosages (00 HPMC capsule, 000 HPMC capsule, and H2-Hydrogel from powder scoop) measured in mPa·s as a function of number of units.

### DETAILED DESCRIPTION

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Provided herein are compositions and methods to generate molecular hydrogen in aqueous acidic compositions. The compositions provided herein maintain levels of molecular hydrogen (H₂) within the composition for longer periods of time and release the molecular hydrogen more slowly than in other compositions previously known in the art.

Molecular hydrogen (H2) has been shown to modulate signal transduction, protein phosphorylation cascades, gene expression, autophagy, miRNA expression, as well as other metabolic processes. H2 has been proposed to act as an exercise mimetic and redox modulator given its ability to attenuate oxidative stress. However, most H2 studies have been conducted using relatively low concentrations of H2, or relatively short-term exposure to H2. In addition to the H2 concentration being important, the duration of use can impact the effects following administration. Since awareness of the relevance of the H2 concentration and the duration of use has increased, high-concentration H2 produced via magnesium is gaining popularity as compared to low-concentration H2. High-concentration H2 produced via magnesium is more effective in activating the NRF2 pathway, which leads to increased destruction of ROS (reactive oxygen species) by catalase, superoxide dismutase, and glutathione peroxidase. Given H2's potential use for wellness, anti-aging, as well as prevention and treatment of numerous inflammatory and metabolic diseases novel methods of stable dose administration are needed.

Disclosed herein is the surprising discovery that molecular hydrogen administration can be controlled with regards to concentration and pace of administration via diffusion. Following oral administration, a composition comprising glucomannan, polysaccharide gum, and magnesium metal powder and immersed in aqueous acidic solution releases H2. The released H2 is trapped in an expanding mass or hydrogel comprising glucomannan, polysaccharide gum, and magnesium metal powder. The expanding mass or hydrogel diffuses H2 for an extended period of time within the stomach of the individual. Further disclosed herein are embodiments of the composition and methods of using the same.

The section headings used herein are for organizational purposes only and are not to be construed as limiting the subject matter described.

### I. Generation of Molecular Hydrogen

The concentration of hydrogen (H₂) is often reported in molarity (moles/liter (M) or millimoles/L (mM)), parts per million (ppm), parts per billion (ppb) or milligrams per liter (mg/L). In dilute concentrations, 1 ppm is about the same as 1 mg/L and they are often used interchangeably. The molar mass of molecular hydrogen is about 2 mg/millimole, 1 mg is approximately equivalent to 0.5 moles, therefore 1 ppm=1 mg/L=0.5 mM.

The solubility of gas dissolved in water is a function of pressure and temperature according to Henry's law:

C=P/K_{H}

where C represents the concentration of the dissolved gas (mol/L), K_{H} is a constant characteristic of the particular gas (Latm/mol), and P represents the partial pressure of the specific gas above the solution (atm). Given hydrogen (H₂) gas constitutes 5.5×10⁻⁵% of atmosphere, and Henry's Constant at 25 C is 1282.05 l*atm/mol, the concentration of hydrogen in water at 1 atmosphere is 4.29×10⁻⁷ mM, 8.65×10⁻⁷, or 8.65×10⁻⁴ ppb.

Generally known methods of generating molecular hydrogen for health care purposes include: electrolysis of water, reaction of base metals and metal hydrides with water, direct water splitting through vibrating piezoelectric zinc oxide microfibers in aqueous solutions, and pressurizing molecular hydrogen into water in containers resistant to permeation.

In some embodiments described herein, the molecular hydrogen is generated using electrolysis. Several commercial electrolysis devices are available for generating molecular hydrogen from water. These devices are limited in that they require ‘purified’ water. ‘Purified’ water is defined here as water that is free of contaminants. Of most concern is the presence of those electrolytes in water that can form deposits on the electrodes of the electrolysis device and render it useless. ‘Purified’ water can be produced by distillation, reverse osmosis, ion exchange or any method that results in water that is free of, or very low in, ions and organic contaminants. For electrolysis, the pH should be near neutral and free of electrolytes that can degrade the electrodes. Use of electrolytes greatly reduces the lifetime of electrodes. A major disadvantage in using ‘purified’ water, e.g., distilled water, is the very low output of H2. To increase molecular hydrogen generation from ‘purified’ water, high voltage, unsafe for consumers, is generally used.

In some embodiments described herein, the molecular hydrogen is generated from a composition comprising magnesium metal powder, glucomannan, polysaccharide gum, organic acid, excipients, and any combination thereof. In some embodiments, the molecular hydrogen is generated from a composition comprising magnesium metal powder, glucomannan, and polysaccharide gum. In some embodiments, the molecular hydrogen is generated from a composition comprising magnesium metal powder, glucomannan, and xantham gum. In some embodiments, the molecular hydrogen is generated from a composition comprising magnesium metal powder, glucomannan, and a polysaccharide gum wherein the magnesium metal in the composition reacts with an aqueous solution to produce molecular hydrogen prior to oral co-administration with food to an individual. In some embodiments, the molecular hydrogen is generated from a composition comprising magnesium metal powder, glucomannan, and a polysaccharide gum wherein the magnesium metal in the composition reacts with an acidic solution to produce molecular hydrogen following oral co-administration with food to an individual.

### II. Glucomannan

Konjac glucomannan is derived from the tuber of the Amorphophallus konjac plant, which is prevalent in Asian countries including China, Indonesia, and Japan. Glucomannan is used in preparing several types of foods. Glucomannan flour contains a variety of insoluble substances as well as water-soluble substances. Glucomannan is a polymer composed of the monosaccharides D-glucose and D-mannose. It forms a highly viscous sol when constituted with water at concentrations of pure glucomannan above 1.0% w/w. It is the only biopolymer currently known to form an aqueous gel at room temperature. The gel forms within a few minutes of mixing with water. U.S. Pat. No. 5,486,364 describes processes for preparing konjac glucomannan and is incorporated herein for such disclosure.

U.S. patent application Ser. No. 16/73,6749 and PCT/US2019/042833 teach that glucomannan has a remarkable ability to sequester molecular hydrogen (H2) and, when forming an aqueous gel, the H2 markedly affects a volume expansion of the gel by up to 70%—above that occupied by a non-gelling aqueous solution- and is incorporated herein for such disclosure. Further, U.S. patent application Ser. No. 16/73,6749 and PCT/US2019/042833 teach that the extent of the expansion being dependent upon factors such as the concentration of glucomannan, the amount of H2 present and the acidity of the aqueous media in which the components are present, and is incorporated herein for such disclosure.

In some embodiments, compositions described herein comprise clarified glucomannan that forms a clear sol with water. In some embodiments, compositions described herein comprise rapidly hydratable konjac glucomannan, glucomannan, or glucomannan (NOW® Foods) that is characterized by at least a 60% viscosity gain after a 10 minute period. In some embodiments, compositions described herein comprise chemically modified glucomannans.

Glucomannan has been used in Asia, particularly in China, for over 2,000 years in applications for detoxification, tumor suppression, blood stasis alleviation and to treat ailments such as asthma, cough, hernia, breast pain, burns as well as hematological and skin disorders. Glucomannan has additionally been shown to affect body weight reduction in animal and human studies.

In some embodiments, the composition comprises from about 2% w/v to about 98% w/v glucomannan. In some embodiments, the composition comprises glucomannan from about 40% w/v to about 50% w/v. In some embodiments, the composition comprises glucomannan from about 2% w/v to about 5% w/v, about 2% w/v to about 10% w/v, about 2% w/v to about 20% w/v, about 2% w/v to about 30% w/v, about 2% w/v to about 40% w/v, about 2% w/v to about 50% w/v, about 2% w/v to about 60% w/v, about 2% w/v to about 70% w/v, about 2% w/v to about 80% w/v, about 2% w/v to about 90% w/v, about 2% w/v to about 98% w/v, about 5% w/v to about 10% w/v, about 5% w/v to about 20% w/v, about 5% w/v to about 30% w/v, about 5% w/v to about 40% w/v, about 5% w/v to about 50% w/v, about 5% w/v to about 60% w/v, about 5% w/v to about 70% w/v, about 5% w/v to about 80% w/v, about 5% w/v to about 90% w/v, about 5% w/v to about 98% w/v, about 10% w/v to about 20% w/v, about 10% w/v to about 30% w/v, about 10% w/v to about 40% w/v, about 10% w/v to about 50% w/v, about 10% w/v to about 60% w/v, about 10% w/v to about 70% w/v, about 10% w/v to about 80% w/v, about 10% w/v to about 90% w/v, about 10% w/v to about 98% w/v, about 20% w/v to about 30% w/v, about 20% w/v to about 40% w/v, about 20% w/v to about 50% w/v, about 20% w/v to about 60% w/v, about 20% w/v to about 70% w/v, about 20% w/v to about 80% w/v, about 20% w/v to about 90% w/v, about 20% w/v to about 98% w/v, about 30% w/v to about 40% w/v, about 30% w/v to about 50% w/v, about 30% w/v to about 60% w/v, about 30% w/v to about 70% w/v, about 30% w/v to about 80% w/v, about 30% w/v to about 90% w/v, about 30% w/v to about 98% w/v, about 40% w/v to about 50% w/v, about 40% w/v to about 60% w/v, about 40% w/v to about 70% w/v, about 40% w/v to about 80% w/v, about 40% w/v to about 90% w/v, about 40% w/v to about 98% w/v, about 50% w/v to about 60% w/v, about 50% w/v to about 70% w/v, about 50% w/v to about 80% w/v, about 50% w/v to about 90% w/v, about 50% w/v to about 98% w/v, about 60% w/v to about 70% w/v, about 60% w/v to about 80% w/v, about 60% w/v to about 90% w/v, about 60% w/v to about 98% w/v, about 70% w/v to about 80% w/v, about 70% w/v to about 90% w/v, about 70% w/v to about 98% w/v, about 80% w/v to about 90% w/v, about 80% w/v to about 98% w/v, or about 90% w/v to about 98% w/v. In some embodiments, the composition comprises glucomannan from about 2% w/v, about 5% w/v, about 10% w/v, about 20% w/v, about 30% w/v, about 40% w/v, about 50% w/v, about 60% w/v, about 70% w/v, about 80% w/v, about 90% w/v, or about 98% w/v. In some embodiments, the composition comprises glucomannan from at least about 2% w/v, about 5% w/v, about 10% w/v, about 20% w/v, about 30% w/v, about 40% w/v, about 50% w/v, about 60% w/v, about 70% w/v, about 80% w/v, or about 90% w/v. In some embodiments, the composition comprises glucomannan from at most about 5% w/v, about 10% w/v, about 20% w/v, about 30% w/v, about 40% w/v, about 50% w/v, about 60% w/v, about 70% w/v, about 80% w/v, about 90% w/v, or about 98% w/v.

In some embodiments, formulations according to the invention consist essentially of the glucomannan composition. In some embodiments, the present invention provides glucomannan compositions comprising at least one base metal capable of generating molecular hydrogen in acidic conditions. In some embodiments, the base metal capable of generating molecular hydrogen in acidic conditions is magnesium metal powder. In some embodiments, magnesium metal powder releases molecular hydrogen. In some embodiments, formulations according to the invention consist essentially of the glucomannan composition comprising magnesium metal powder capable of releasing molecular hydrogen gas in aqueous acidic solutions. In some embodiments, formulations according to the invention consist essentially of the glucomannan composition comprising magnesium metal powder is capable of releasing molecular hydrogen gas in aqueous acidic solutions following co-administration with food.

### III. Xanthan Gum

Gums also known as hydrocolloids or polysaccharides, are very versatile biopolymers or complex carbohydrates. Gums or polysaccharides are soluble in water and can form gels and mucilages. Gums or polysaccharides are extensively used in the food sector as ingredient or additive, which fulfill several technological and, sometimes, nutritional functions. This versatility is intrinsically related to their molecular composition, which gives these polysaccharides certain properties such as gelling, thickening, moisture retention, emulsification, and stabilization. In the food industry, gums or polysaccharides are widely used in confectionery, as ice cream stabilizers, food emulsions, in the microencapsulation of flavors and dyes, clarifiers, and beverage stabilizers.

Classified according to origin, behavior, and chemical structure, gums can be derived from plant seed endosperm (guar gum), plant exudates (tragacanth), shrubs or trees (gum arabic, karaya gum, cashew gum), algae extracts (agar, alginate, carrangeenan), bacteria (xanthan gum), animal source (chitin), and others. Gums or polysaccharides have high molar mass and can be formed by galactose, arabinose, rhamnose, xylose, galacturonic acid, or any combination thereof.

Xanthan gum (XG) is a generally recognized as safe (GRAS) polysaccharide that is used in preparing several types of ingestibles or food items. Xanthan gum is commonly used as a food additive. Xanthan gum is commonly used as a thickener of food. Xanthan gum is also commonly used to prevent ingredients from separating. Xanthan gum also suspends solid particles, and at 1% w/v, produces a modest increase in the viscosity of an aqueous solution. Xanthan gum does not change the color or flavor of foods or beverages. Xanthan gum is used in wide range food products, such as sauces, dressings, meat and poultry products, bakery products, confectionery products, beverages, and dairy products.

In some embodiments, the composition comprises a gum or polysaccharide wherein the gum or polysaccharide is xanthan gum, carrageenan, agar, and alginate. In some embodiments, the composition comprises a gum or polysaccharide wherein the gum or polysaccharide is xanthan gum. In some embodiments, the composition comprises xanthan gum from about 0% w/v to about 98% w/v. In some embodiments, the composition comprises xanthan gum from about 40% w/v to about 50% w/v. In some embodiments, the composition comprises xanthan gum from about 0% w/v to about 5% w/v, about 0% w/v to about 10% w/v, about 0% w/v to about 20% w/v, about 0% w/v to about 30% w/v, about 0% w/v to about 40% w/v, about 0% w/v to about 50% w/v, about 0% w/v to about 60% w/v, about 0% w/v to about 70% w/v, about 0% w/v to about 80% w/v, about 0% w/v to about 90% w/v, about 0% w/v to about 98% w/v, about 5% w/v to about 10% w/v, about 5% w/v to about 20% w/v, about 5% w/v to about 30% w/v, about 5% w/v to about 40% w/v, about 5% w/v to about 50% w/v, about 5% w/v to about 60% w/v, about 5% w/v to about 70% w/v, about 5% w/v to about 80% w/v, about 5% w/v to about 90% w/v, about 5% w/v to about 98% w/v, about 10% w/v to about 20% w/v, about 10% w/v to about 30% w/v, about 10% w/v to about 40% w/v, about 10% w/v to about 50% w/v, about 10% w/v to about 60% w/v, about 10% w/v to about 70% w/v, about 10% w/v to about 80% w/v, about 10% w/v to about 90% w/v, about 10% w/v to about 98% w/v, about 20% w/v to about 30% w/v, about 20% w/v to about 40% w/v, about 20% w/v to about 50% w/v, about 20% w/v to about 60% w/v, about 20% w/v to about 70% w/v, about 20% w/v to about 80% w/v, about 20% w/v to about 90% w/v, about 20% w/v to about 98% w/v, about 30% w/v to about 40% w/v, about 30% w/v to about 50% w/v, about 30% w/v to about 60% w/v, about 30% w/v to about 70% w/v, about 30% w/v to about 80% w/v, about 30% w/v to about 90% w/v, about 30% w/v to about 98% w/v, about 40% w/v to about 50% w/v, about 40% w/v to about 60% w/v, about 40% w/v to about 70% w/v, about 40% w/v to about 80% w/v, about 40% w/v to about 90% w/v, about 40% w/v to about 98% w/v, about 50% w/v to about 60% w/v, about 50% w/v to about 70% w/v, about 50% w/v to about 80% w/v, about 50% w/v to about 90% w/v, about 50% w/v to about 98% w/v, about 60% w/v to about 70% w/v, about 60% w/v to about 80% w/v, about 60% w/v to about 90% w/v, about 60% w/v to about 98% w/v, about 70% w/v to about 80% w/v, about 70% w/v to about 90% w/v, about 70% w/v to about 98% w/v, about 80% w/v to about 90% w/v, about 80% w/v to about 98% w/v, or about 90% w/v to about 98% w/v. In some embodiments, the composition comprises xanthan gum from about 0% w/v, about 5% w/v, about 10% w/v, about 20% w/v, about 30% w/v, about 40% w/v, about 50% w/v, about 60% w/v, about 70% w/v, about 80% w/v, about 90% w/v, or about 98% w/v. In some embodiments, the composition comprises xanthan gum from at least about 0% w/v, about 5% w/v, about 10% w/v, about 20% w/v, about 30% w/v, about 40% w/v, about 50% w/v, about 60% w/v, about 70% w/v, about 80% w/v, or about 90% w/v. In some embodiments, the composition comprises xanthan gum from at most about 5% w/v, about 10% w/v, about 20% w/v, about 30% w/v, about 40% w/v, about 50% w/v, about 60% w/v, about 70% w/v, about 80% w/v, about 90% w/v, or about 98% w/v.

### IV. Magnesium Metal Powder

Several base metals, for example, lithium, potassium, strontium, calcium, sodium, magnesium metal powder, aluminum, zinc, chromium, manganese and iron, when reacting with water, generate molecular hydrogen. Conditions such as temperature, or the presence of acids, bases, and other catalysts can affect the rate of the reaction. Magnesium metal powder is preferred for human consumption due to its wide margin of safety, established health benefits as an essential element and ease of molecular hydrogen generation under room temperature, atmospheric pressure, and mild acidic or basic conditions.

In some embodiments described herein, compositions comprise lithium, potassium, strontium, calcium, sodium, magnesium metal powder, aluminum, zinc, chromium, manganese, iron, or any combination thereof. In some embodiments, compositions comprise lithium. In some embodiments, compositions comprise potassium. In some embodiments, compositions comprise strontium. In some embodiments, compositions comprise calcium. In some embodiments, compositions comprise sodium. In some embodiments, compositions comprise magnesium metal powder. In some embodiments, compositions comprise aluminum. In some embodiments, compositions comprise zinc. In some embodiments, compositions comprise chromium. In some embodiments, compositions comprise iron.

In embodiments described herein, compositions comprise from about 0.001% w/v to about 30% w/v of the base metal. In embodiments described herein, compositions comprise from about 0.001% w/v to about 5% w/v of the base metal. In embodiments described herein, compositions comprise from about 0.001% w/v to about 2% w/v of the base metal. In some embodiments, compositions comprise from about 0.001% w/v to about 0.01% w/v of the base metal. In some embodiments, compositions comprise from about 0.005% w/v to about 0.05% w/v of the base metal. In some embodiments, compositions comprise from about 0.01% w/v to about 0.1% w/v of the base metal. In some embodiments, compositions comprise from about 0.05% w/v to about 0.5% w/v of the base metal. In some embodiments, compositions comprise from about 0.1% w/v to about 1% w/v of the base metal. In some embodiments, compositions comprise from about 0.5% w/v to about 2% w/v of the base metal.

In some embodiments, compositions described herein comprise magnesium metal powder (Mg). In some embodiments, the composition comprises magnesium metal powder from about 0% w/v to about 30% w/v. In some embodiments, the composition comprises magnesium metal powder from about 0% w/v to about 5% w/v. In some embodiments, the composition comprises magnesium metal powder from about 0% w/v to about 5% w/v, about 0% w/v to about 10% w/v, about 0% w/v to about 15% w/v, about 0% w/v to about 20% w/v, about 0% w/v to about 25% w/v, about 0% w/v to about 30% w/v, about 5% w/v to about 10% w/v, about 5% w/v to about 15% w/v, about 5% w/v to about 20% w/v, about 5% w/v to about 25% w/v, about 5% w/v to about 30% w/v, about 10% w/v to about 15% w/v, about 10% w/v to about 20% w/v, about 10% w/v to about 25% w/v, about 10% w/v to about 30% w/v, about 15% w/v to about 20% w/v, about 15% w/v to about 25% w/v, about 15% w/v to about 30% w/v, about 20% w/v to about 25% w/v, about 20% w/v to about 30% w/v, or about 25% w/v to about 30% w/v. In some embodiments, the composition comprises magnesium metal powder from about 0% w/v, about 5% w/v, about 10% w/v, about 15% w/v, about 20% w/v, about 25% w/v, or about 30% w/v. In some embodiments, the composition comprises magnesium metal powder from at least about 0% w/v, about 5% w/v, about 10% w/v, about 15% w/v, about 20% w/v, or about 25% w/v. In some embodiments, the composition comprises magnesium metal powder from at most about 5% w/v, about 10% w/v, about 15% w/v, about 20% w/v, about 25% w/v, or about 30% w/v.

Organic acids have been shown to be effective in accelerating the generation of molecular hydrogen in the reaction of magnesium metal powder with water. (See, Uan, J-Y. et. al. (2009) J. of Hydrogen Energy 34 (15), 6137-6142, which is incorporated herein for such disclosure). Organic acids can include, without limitation, lactic acid, acetic acid, formic acid, citric acid, oxalic acid, uric acid, malic acid, succinic acid, tartaric acid, adipic acid, or any combination thereof. In some embodiments, compositions comprise lactic acid. In some embodiments, compositions comprise acetic acid. In some embodiments, compositions comprise formic acid. In some embodiments, compositions comprise citric acid. In some embodiments, compositions comprise oxalic acid. In some embodiments, compositions comprise uric acid. In some embodiments, compositions comprise malic acid.

### V. Commercial Products Containing Magnesium Metal Powder

Products based upon magnesium metal powder, are known in the art for generating molecular hydrogen when reacted with acidic water. Such products require acids, elevated temperature, or catalysts to rapidly generate molecular hydrogen from water. These products do not have a means of sustaining molecular hydrogen in solution or in the body and do not form hydrogels.

### VI. Rationale for Use of Magnesium Metal Powder

In some embodiments described herein, compositions comprise magnesium metal powder. Magnesium metal powder is safe to use for human and animal consumption. It is stable, readily generates molecular hydrogen, is inexpensive, and is commercially available. When reacting with water, it is converted to Generally Recognized as Safe (GRAS) magnesium hydroxide according the reaction

Mg+2H₂O→Mg(OH)₂+H₂.

Magnesium hydroxide is an OTC drug approved laxative as well as approved as a GRAS food additive and supplement. Magnesium is an essential nutrient involved in over 400 enzymatic reactions in living systems. Magnesium metal reacts with water to produce molecular hydrogen and form magnesium hydroxide. This reaction is thermodynamically favorable, and its reaction rate is pH and temperature-dependent. Ionized magnesium in a salt form, as non-limiting examples, citrate, chloride, sulfate, or chelated magnesium, cannot generate molecular hydrogen in an aqueous environment. Further, covalently bound magnesium compounds such as magnesium oxide, magnesium hydroxide, magnesium carbonate cannot generate molecular hydrogen in an aqueous environment. The rate of reaction of magnesium metal powder with water depends on several factors, some of which are discussed below. The surface area of magnesium metal that will be exposed to water is a factor to consider. Basic physical chemistry would predict that the larger the surface area, the faster and more efficient the reaction. This otherwise obvious conclusion is modified by two factors: First, the larger the surface area and smaller the particle size, the more likely that magnesium metal can undergo a spontaneous and explosive reaction with oxygen. Thus, safety is an issue. Secondly, left alone or by a process of reducing the risk of spontaneous reaction with oxygen (or water), a magnesium oxide coat is spontaneously formed on the surface of magnesium metal particles through a process called passivation. Although this coat of magnesium oxide does not greatly reduce the amount of magnesium metal in large particles that can react with water, it potentially can reduce the amount of magnesium in small particles that can react with water. For example, the magnesium oxide coat on nanoparticles may constitute a significant percentage of the magnesium present—reducing the amount of magnesium metal available to react with water. All forms of magnesium metal, including but not limited to powders, pellets, and filings, will have some reactivity with water and oxygen. The point to be taken is that there is a particle size distribution of magnesium metal powder that is optimally reactive and safe to handle under Good Manufacturing Practice (GMP) conditions. Magnesium metal powder is generally available at up to 99.999% purity, with aluminum being the main impurity.

The size distribution of magnesium metal powder used in the studies described herein was determined using Fieldmaster® Sieves of 35, 60, 120, and 230 Mesh, corresponding, respectively to 500, 250, 125 and 63 microns. The size distribution was found to be: 0.7% equal to or greater than 500 microns; 1.2% 250-500 microns; 9.3% 125-250 microns; 38.2% at 63-125 microns, and 50.9% smaller than 63 microns.

### VII. Magnesium Metal Powder Plus Glucomannan Powder

The reaction rate of magnesium metal powder with water in the presence of glucomannan is described herein, and depends on several factors, including pH, temperature, and the presence of catalysts. For forming gels using magnesium metal powder-glucomannan complexes, a fast rate of molecular hydrogen production is not necessarily desirable. That is, more molecular hydrogen will be sequestered in the gel if the gel is formed before a significant percentage of the available molecular hydrogen is generated.

As is well known in the art, variation of pH from neutrality can accelerate the reaction of magnesium metal with water. Acidic conditions can include any organic or inorganic acid that lowers the pH below 7.0. Included are gastric fluids, acidic foods, and acidic aquatic environments. Examples of acids and/or their salts that can be used include citric acid, malic acid, adipic acid, fumaric acid, succinic acid, ascorbic acid, iso-ascorbic acid, salicylic acid, phosphoric acid, potassium sorbate and sodium bisulfite. Examples of antioxidants, that are salts of acids, include sodium ascorbate and potassium ascorbate. On the alkaline side, magnesium oxide, magnesium hydroxide, potassium and sodium hydroxide can be used to increase the pH to alkaline conditions. Combinations of these agents can be used to control the rates of reaction of magnesium metal in aqueous glucomannan at conditions that generate H₂ in solutions and gels.

In some embodiments described herein, compositions comprise an acid or antioxidant catalyst. In some embodiments, the compositions are formulated for oral administration for gastric delivery. In some embodiments, the oral formulation comprises capsules, powder, tablets or another delivery vehicle. In some embodiments, the composition formulation for gastric delivery utilizes the acidity of stomach fluid to catalyze the reaction of magnesium metal or a magnesium hydride with water to form the molecular hydrogen-rich solution or a viscous solution or a gel. Also, when adding a magnesium metal powder-glucomannan formulation to acidic products, such as tea or acidic beverages, the acidity of the product is more than sufficient to catalyze the reaction of water with magnesium metal powder to generate molecular hydrogen. In some embodiments, the composition comprises a base, for example, sodium bicarbonate, to modulate the reaction.

It has been found that when combining magnesium metal powder and glucomannan and mixing with water—that a hydrogen-rich solution, viscous solution or expansive gel with unexpected high sustainability of molecular hydrogen is created.

Depending on the concentrations of magnesium metal powder and glucomannan, the pH, temperature, and activity of water—the resulting solutions and gel-like structures display the following properties:
1. When magnesium metal powder reacts with acidic water in the presence of 40-50% glucomannan, it generates molecular hydrogen which expands the resultant hydrogel by up to 300 times the original volume of the composition.
2. H₂ sequestration by magnesium metal powder—glucomannan gels creates aqueous gels of various density and porosity thereby affecting a desirable ‘fluffy’ texture—depending of the concentration of the basic constituents.
3. Great tasting, ‘fluffy’ textured, nutritious aqueous magnesium metal powder—glucomannan gels, with health benefits, can be created by incorporating one or more of the following into the gels: sweeteners, flavoring agents, natural fruit and vegetable powders, tea powders, protein powders, vitamin powders, probiotic powders, prebiotics, drugs and nutritional supplements.
4. Exposing aqueous solutions or gels of glucomannan to molecular hydrogen allows the sequestration of molecular hydrogen by glucomannan.
5. Long lasting H₂ microbubble stability in non-viscous magnesium metal powder—glucomannan solutions, in an open system, at room temperature.
6. Creation of H₂ sustained release gels, of various rates of release, for nutrients, drugs and beneficial environmental factors, including H2.

Furthermore, it has been conceived that the resulting molecular hydrogen—glucomannan gels and viscous solutions can be utilized to:
1. Provide extended, long lasting delivery of molecular hydrogen to the body of mammals through its persistence in the gastrointestinal tract and other tissues—such as skin;
2. Use of this biotechnology to produce hydrogen-rich magnesium metal powder-glucomannan gels that are low calorie, good tasting, filling, and long lasting due to floating and expanding in the upper G-I tract—properties that are useful for augmentation of fasting, anti-inflammatory, and weight control programs.
3. Provide molecular hydrogen—generating capsules or tablets, taken orally, that support weight control and fasting regimens by forming an expanded, floating gel in the aqueous acidic environment of the stomach—while reducing the inflammatory complications of obesity.
4. Provide extended, long lasting delivery of molecular hydrogen (H₂) to the oral cavity of humans and animals to treat plaque build-up, gingivitis, periodontal disease, and other inflammatory conditions of the oral cavity, esophagus and stomach.

### VIII. Encapsulated Compositions of Magnesium Metal Powder Plus Glucomannan Powder and Xanthan Gum

It has been found that when combining magnesium metal powder, glucomannan and xanthan gum, and mixing with acidic water—that a hydrogen-rich solution, viscous solution or expansive gel with unexpected high sustainability of molecular hydrogen is created. Depending on the concentrations of magnesium metal powder and glucomannan, the pH, temperature, acidity of the aqueous acidic solution, and the administration of the composition—the resulting solutions and gel-like structures display the following properties, new findings, special benefits, and advantages:
1. Encapsulated mixtures of glucomannan (GMN):xanthan gum (XG), when immersed in an aqueous acidic solution, form low viscosity gels with no volume expansion.
2. Encapsulating glucomannan with magnesium metal powder and immersing the capsules in aqueous acidic environment, as found in the stomach, provides for superior gelation as compared to immersing a capsule containing glucomannan, alone, in the same solution.
3. Encapsulating glucomannan and magnesium metal powder with xanthan gum and then immersing in an acidic solution, provides for superior gelation and volume expansion from the capsule—compared to when encapsulated glucomannan and magnesium metal powder are tested under the same conditions.
4. Containing, in capsule form, the glucomannan, magnesium metal powder, with- or without xanthan gum was not disclosed in Ser. No. 16/73,6749 and PCT/US2019/042833.
5. Orally administering encapsulated glucomannan, xanthan gum and magnesium metal powder to individuals affects satiety.
6. Allowing molecular hydrogen to affect the dissolution of the capsule containing GMN:XG:MMP and expand its volume by 100-200 times the volume of the capsules—in the process of gelling the aqueous-acidic solution.
7. Ingesting the GMN/XG/MMP capsules, with food facilitates the dissolution of the contents of the capsules.
8. Surprisingly uncovering that delivering GMN:MMP from a capsule to an acidic solution, contracts the resultant gel by 7-9%. In comparison, delivering a free-flowing powder containing GMN:MMP to an acidic solution—expands the gel volume up to 70% above that occupied by a non-gelling aqueous solution.
9. Dispersing a GMN/XG/MMP/organic acid powder, in water, affects a larger hydrogen volume and higher viscosity than can be achieved with immersing, in acidic water, capsules containing GMN/XG/MMP.

### IX. Magnesium Metal Powder—Glucomannan Gels with Excipients and Functional Agents

In some embodiments described herein, compositions comprise additional excipients or functional agents. Excipients or functional agent included in some compositions described herein comprise sweeteners, flavoring agents, natural fruit and vegetable powders, tea powders, protein powders, vitamin powders, probiotic powders, anti-caking agents, preservatives, prebiotics, nutritional supplements, drugs, and food colorings.

In some embodiments, compositions comprise sweeteners, alone or in combination, including, but not limited to, acesulfame, aspartame, saccharin, sucralose, sucrose, monk fruit, glucose, fructose, xylitol, mannitol, glycerin, maltodextrin, inulin, and erythritol. Compositions described herein may contain sweeteners, alone or in combination, in a concentration of from about 0.1 to about 20% w/w of the composition. Compositions described herein may contain sweeteners, alone or in combination, in a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 12%, about 14%, about 16%, about 18%, about 20%, from about 0.1% to about 5%, from about 1% to about 10%, from about 5% to about 15%, or from about 10% to about 20%, or from about 0.1% to about 20% w/w of the composition.

In some embodiments, compositions comprise flavoring agents, alone or in combination, including, but not limited to, natural lemon powder, citric acid, malic acid, hydroxy-citric acid, tartaric acid, adipic acid, vanillin, chocolate, cherry, pomegranate, raspberry, and strawberry flavoring. Compositions described herein may contain flavoring agents, alone or in combination, in a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 12%, about 14%, about 16%, about 18%, about 20%, %, about 25%, about 30%, about 35%, about 40%, about 45%, about 50%, from about 0.1% to about 5%, from about 1% to about 10%, from about 5% to about 15%, or from about 10% to about 20%, from about 15% to about 25%, from about 20% to about 30%, from about 25% to about 35%, from about 30% to about 40%, from about 35% to about 45%, from about 40% to about 50%, or from about 0.1% to about 50% w/w of the composition.

In some embodiments, compositions comprise water extracts of natural fruit powders, alone or in combination, including, but not limited to, bilberry, lemon, blueberry, cranberry, cinnamon, ginger, lemon balm, vanilla, pumpkin seed and strawberry. Compositions described herein may contain water extracts of natural fruit powders, alone or in combination, in a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 12%, about 14%, about 16%, about 18%, about 20%, %, about 25%, about 30%, about 35%, about 40%, about 45%, about 50%, from about 0.1% to about 5%, from about 1% to about 10%, from about 5% to about 15%, or from about 10% to about 20%, from about 15% to about 25%, from about 20% to about 30%, from about 25% to about 35%, from about 30% to about 40%, from about 35% to about 45%, from about 40% to about 50%, or from about 0.1% to about 50% w/w of the composition.

In some embodiments, compositions comprise water extracts of natural vegetable powders, alone or in combination, including, but not limited to, carrot juice, spinach, broccoli, sweet potato and white willow bark powder. Compositions described herein may contain water extracts of natural vegetable powders, alone or in combination, in a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 12%, about 14%, about 16%, about 18%, about 20%, %, about 25%, about 30%, about 35%, about 40%, about 45%, about 50%, from about 0.1% to about 5%, from about 1% to about 10%, from about 5% to about 15%, or from about 10% to about 20%, from about 15% to about 25%, from about 20% to about 30%, from about 25% to about 35%, from about 30% to about 40%, from about 35% to about 45%, from about 40% to about 50%, or from about 0.1% to about 50% w/w of the composition.

In some embodiments, compositions comprise herbal tea water extract powders, alone or in combination, including, but not limited to, white tea, green tea, Red Gush Chai, Matcha, Maca, Kombucha, Turmeric, Dandelion, Ginger, Lemon Ginger, Oolong, Rooibos, Fennell, Nettle Leaf, Peppermint, Echinacea, Valerian, Cinnamon Berry, Chamomile and Lavender tea. Compositions described herein may contain herbal tea water extract powders, alone or in combination, in a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 12%, about 14%, about 16%, about 18%, about 20%, %, about 25%, about 30%, about 35%, about 40%, about 45%, about 50%, about 55%, about 60%, about 65%, about 70%, from about 0.1% to about 5%, from about 1% to about 10%, from about 5% to about 15%, or from about 10% to about 20%, from about 15% to about 25%, from about 20% to about 30%, from about 25% to about 35%, from about 30% to about 40%, from about 35% to about 45%, from about 40% to about 50%, from about 45% to about 55%, from about 50% to about 60%, from about 55% to about 65%, from about 60% to about 70%, or from about 0.1% to about 70% w/w of the composition.

In some embodiments, compositions comprise protein powders, alone or in combination, including, but are not limited to, non-fat milk, 1% fat milk, 2% fat milk, whole milk, goat milk, rice milk, almond milk, soy milk, coconut, pea protein and brown rice protein. Compositions described herein may contain protein powders, alone or in combination, in a concentration of about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 12%, about 14%, about 16%, about 18%, about 20%, %, about 25%, about 30%, about 35%, about 40%, about 45%, about 50%, from about 0.2% to about 5%, from about 1% to about 10%, from about 5% to about 15%, or from about 10% to about 20%, from about 15% to about 25%, from about 20% to about 30%, from about 25% to about 35%, from about 30% to about 40%, from about 35% to about 45%, from about 40% to about 50%, or from about 0.2% to about 50% w/w of the composition.

In some embodiments, compositions comprise vitamins or minerals, alone or in combination, including, but are not limited to, vitamin A, vitamin C, calcium, iron, vitamin D3, vitamin E, thiamin, riboflavin, niacin, vitamin B6, Folate, vitamin B12, biotin, pantothenic acid, phosphorous, iodine, magnesium metal powder, zinc, selenium, copper, manganese and chromium. Compositions described herein may contain vitamins or minerals, alone or in combination, in an amount of about 5%, about 6%, about 7%, about 8%, about 9%, about 10%, about 12%, about 14%, about 16%, about 18%, about 20%, %, about 25%, about 30%, about 35%, about 40%, about 45%, about 50%, about 55%, about 60%, about 65%, about 70%, about 75%, about 80%, about 85%, about 90%, about 95%, about 100%, from about 5% to about 15%, or from about 10% to about 20%, from about 15% to about 25%, from about 20% to about 30%, from about 25% to about 35%, from about 30% to about 40%, from about 35% to about 45%, from about 40% to about 50%, from about 45% to about 55%, from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 100%, or from about 5% to about 100% of the RDI (Required Daily Intake) for a healthy adult human.

In some embodiments, compositions comprise probiotics, alone or in combination, including, but not limited to Bacillus coagulans, Bacillus subtilis, Bacillus infantis, Lactobacillus longum, Lactobacillus casei, Lactobacillus acidophilus and Bifidobacterium. Compositions described herein may contain probiotics, alone or in combination, in an amount of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, from about 0.1% to about 5%, from about 1% to about 10%, from about 5% to about 15%, or from about 10% to about 20%, from about 15% to about 25%, from about 20% to about 30%, from about 25% to about 35%, from about 30% to about 40%, from about 35% to about 45%, from about 40% to about 50%, or from about 0.1% to about 10% w/w of the composition.

In some embodiments, compositions comprise anti-caking agents, alone or in combination, including, but not limited to, calcium phosphate tribasic, calcium silicate, sodium alginate, cellulose, microcrystalline cellulose, xanthan gum, magnesium carbonate, magnesium oxide, magnesium silicate, and magnesium sulfate. Compositions described herein may contain anti-caking agents, alone or in combination, in a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, from about 0.1% to about 2%, from about 0.5% to about 3%, from about 1% to about 4%, from about 2% to about 5%, or from about 0.1% to about 5% w/w of the composition.

In some embodiments, compositions comprise preservatives, alone or in combination, including, but not limited to, ascorbic acid, calcium ascorbate, erythorbic acid, sodium ascorbate, sodium erythorbate, benzoic acid, calcium sorbate, potassium sorbate, sorbic acid, citric acid, L-cysteine, lecithin, tartaric acid and tocopherols. Compositions described herein may contain preservatives, alone or in combination, in a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, from about 0.1% to about 4%, from about 1% to about 7%, from about 3% to about 10%, or from about 0.1% to about 10% w/w of the composition.

In some embodiments, compositions comprise prebiotics, alone or in combination, including, but not limited to, psyllium, rice hulks, chicory root, dandelion greens, apples, bananas, artichokes, leeks, and asparagus. Compositions described herein may contain prebiotics, alone or in combination, in a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 12%, about 14%, about 16%, about 18%, about 20%, %, about 25%, about 30%, about 35%, about 40%, about 45%, about 50%, about 55%, about 60%, about 65%, about 70%, about 75%, from about 0.1% to about 5%, from about 1% to about 10%, from about 5% to about 15%, or from about 10% to about 20%, from about 15% to about 25%, from about 20% to about 30%, from about 25% to about 35%, from about 30% to about 40%, from about 35% to about 45%, from about 40% to about 50%, from about 45% to about 55%, from about 50% to about 60%, from about 55% to about 65%, from about 60% to about 70%, from about 65% to about 75%, or from about 0.1% to about 75% w/w of the composition.

In some embodiments, compositions comprise nutritional supplements, alone or in combination, including, but not limited to, L-arginine, L-ornithine, 5-hydroxytryptophan, acetyl L-tyrosine, acetyl-L carnitine, alpha-lipoic acid, ashwagandha, bacopa, berbine, betaine, biotin, choline, creatine, curcumin, fish oil, flaxseed oil, ginger, ginseng, jiaogulan, kelp, manganese, methyl folate, N-acetyl-cysteine, nattokinase, niacin, quercetin, resveratrol, L-theanine, valerian root, vinpocetine and melatonin. Compositions described herein may contain nutritional supplements, alone or in combination, in a concentration of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 12%, about 14%, about 16%, about 18%, about 20%, %, about 25%, about 30%, from about 0.01% to about 0.5%, from about 0.1% to about 5%, from about 1% to about 10%, from about 5% to about 15%, or from about 10% to about 20%, from about 15% to about 25%, from about 20% to about 30%, or from about 0.01% to about 30% w/w of the composition.

In some embodiments, compositions comprise over the counter (OTC) and prescription (Rx) drugs, alone or in combination, including, but not limited to salicylic acid, trans-retinoic acid, the alpha-hydroxy acids (e.g., lactic acid), benzoyl peroxide, bismuth subsalicylate, metronidazole, tetracycline, erythromycin, proton pump inhibitors, misoprostol, antibiotics, anti-fungal drugs, anti-inflammatories, or antacids. Compositions described herein may contain over the counter (OTC) and prescription (Rx) drugs, alone or in combination, in an amount of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 20%, from about 0.1% to about 5%, from about 1% to about 10%, from about 5% to about 15%, from about 10% to about 20%, or from about 0.1% to about 20% w/w of the composition.

In some embodiments, compositions comprise food colorings, alone or in combination, including, but not limited to FD&C Blue #1, FD&C Blue #2, FD&C Green #3, FD&C Red #3, FD&C Yellow #5, FD&C Yellow #6, riboflavin, annatto, carmine, elderberry juice powder, lycopene, or turmeric. Compositions described herein may contain food colorings, alone or in combination, in an amount of about 0.001 ppm, about 0.002 ppm, about 0.003 ppm, about 0.004 ppm, about 0.005 ppm, about 0.006 ppm, about 0.007 ppm, about 0.008 ppm, about 0.009 ppm, about 0.01 ppm, about 0.02 ppm, about 0.03 ppm, about 0.04 ppm, about 0.05 ppm, about 0.06 ppm, about 0.07 ppm, about 0.08 ppm, about 0.09 ppm, about 0.1 ppm, about 0.2 ppm, about 0.3 ppm, about 0.4 ppm, about 0.5 ppm, about 0.6 ppm, about 0.7 ppm, about 0.8 ppm, about 0.9 ppm, about 1 ppm, about 2 ppm, about 3 ppm, about 4 ppm, about 5 ppm, about 6 ppm, about 7 ppm, about 8 ppm, about 9 ppm, about 10 ppm, about 20 ppm, about 30 ppm, about 40 ppm, about 50 ppm, about 60 ppm, about 70 ppm, about 80 ppm, about 90 ppm, about 100 ppm, about 200 ppm, about 300 ppm, about 400 ppm, about 500 ppm, from about 0.001 ppm to about 0.05 ppm, from about 0.05 ppm to about 0.1 ppm, from about 0.1 ppm to about 0.5 ppm, from about 0.5 ppm to about 1 ppm, from about 0.5 ppm to about 5 ppm, from about 1 ppm to about 10 ppm, from about 5 ppm to about 50 ppm, from about 50 ppm to about 200 ppm, from about 100 ppm to about 500 ppm, or from about 0.001 ppm to about 500 ppm w/w of the composition.

### X. Non-Parenteral Administration of Magnesium Metal Powder—Glucomannan Gels with Excipients and Functional Agents

Organic or inorganic carrier substances suitable for non-parenteral administration which do not deleteriously react with the components of the magnesium metal powder-glucomannan composition can also be included in the formulation. Suitable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like, or any combination thereof. The formulations can be sterilized and, if desired, mixed with auxiliary agents, e.g., lubricants, preservatives, stabilizers, wetting agents, emulsifiers, salts for influencing osmotic pressure, buffers, colorings flavorings, aromatic substances, or any combination thereof.

The compositions of the present invention may also be formulated as suspensions in aqueous, non-aqueous, or mixed media. Aqueous suspensions may further contain substances which increase the viscosity of the suspension including, for example, sodium carboxymethylcellulose, sorbitol and/or dextran. The suspension may also contain stabilizers, preservatives, antioxidants, hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylc cellulose and carboxypropyl cellulose), and synthetic polymers (for example, carbomers, cellulose ethers, and carboxyvinyl polymers), or any combination thereof. Preservatives can include methyl paraben, propyl paraben, quaternary ammonium salts, benzalkonium chloride, esters of p-hydroxybenzoic acid, and boric acid. Antioxidants added to prevent deterioration of the formulation may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The compositions of this invention can be contained or converted in a known manner into the customary formulations, such as capsules, coated capsules, tablets, coated tablets, pills, pellets, syrups, emulsions, suspensions, gels, pastes, and solutions, or any combination thereof, using inert, non-toxic, suitable excipients or solvents. In some embodiments, the composition can be contained within a capsule, a tablet, a gel, a gummy, a jelly, a food, a drink, a liquid, a syrup, or any combination thereof.

The formulations, which may conveniently be presented in unit dosage form, may be prepared according to conventional techniques well known in the pharmaceutical industry. Such techniques include the step of bringing into association the active ingredients with the carrier(s) or excipient(s). In general, the formulations are prepared by uniformly and intimately bringing into association the active ingredients with liquid carriers or finely divided solid carriers or both, and then, if necessary, shaping the product. The active compound should in each case be present in amounts calculated to ensure the desired therapeutic effect. Compositions may be formulated in a conventional manner using additional acceptable carriers or excipients as appropriate. Thus, the composition may be prepared by conventional means with carriers or excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., starch or sodium starch glycolate); or wetting agents (e.g., sodium lauryl sulfate). In some embodiments, the composition of the invention may further comprise hydroxypropyl methyl cellulose (HPMC).

a. Capsules

Also provided are methods for treating an individual comprising administering to the individual an effective amount of a composition according to the invention. The composition can be administered via non-parenteral routes, for example, buccal, oral, or endoscopic routes. In preferred embodiments, the compositions of the invention are administered orally by means of a capsule.

Capsules are well known in the art. They are generally formulated to be solid at room temperature with a melting temperature below the normal human body temperature of 37° C. It is therefore common to formulate capsules with a fat base, such as cocoa butter, which fulfils the above melting point criteria. In some embodiments, the capsule dosage form may also comprise further excipients such as but not limited to binders and adhesives, lubricants, disintegrants, preservatives, colorants and bulking agents. In some embodiments, the capsule comprises a combination of any of the above-mentioned base substances.

In some embodiments, non-parenteral administration of the compositions of the present invention may involve coating materials. In some embodiments, coating materials may facilitate administration without hampering the activity of the product.

In some embodiments, capsules are coated with fatty or oleaginous bases such as cocoa butter, hard fat and hydrogenated vegetable oil, waxes, water-soluble or water-miscible bases such as polyethylene glycols, glycol-surfactant combinations and polyoxyethylene sorbitan fatty acid esters, coconut oil, glycerinated gelatin, hydrogenated oil, polyethylene glycol (PEG), and combinations thereof. Furthermore, coated capsules can be in the form of oral capsules where a free fatty acid containing core is covered by water-soluble compound such as gelatin.

Further, multicompartment capsules, lipid coated capsules, water permeable capsules, capsules with control release properties, capsules with multi-stage delivery system, or any combination thereof, may also be used to formulate the compositions of the present invention.

In some embodiments, the capsule may comprise a single-piece capsule, two-piece capsule, transparent capsule, non-transparent capsule, opaque capsule, slow-release capsule, extended-release capsule, standard-release capsule, rapid-release capsule, quick-release capsule, hard-shell capsule, soft gel capsule, gel capsule, hard gelatin capsule, soft gelatin capsule, animal-based capsule, vegetarian capsule, polysaccharide capsule, cellulose capsule, mucopolysaccharide capsule, tapioca capsule, hydroxypropylmethyl cellulose (HPMC) capsule, pullulan capsule, enteric capsule, uncoated capsule, coated capsule, capsule comprising titanium dioxide, fatty acids, waxes, shellac, plastics, pasticizers, glycerin, sorbitol, plant fibers, additives, preservatives, colorants, or any combination thereof. In some embodiments, the capsule is a hard-shell capsule.

In some embodiments, hard-shell capsules are manufactured in two halves: a smaller diameter cylinder, close on one end that receives the ingredients and a shorter, but slightly larger piece that is also sealed closed on one end. In some embodiments, the slightly larger piece of the hard-shell capsule is slipped over the open end of the smaller piece of the hard-shell capsule containing the ingredients. In some embodiments, slipping the slightly larger piece over the smaller piece of the hard-shell capsule seals the capsule. The process of encapsulation of the capsules disclosed herein can be done via manual, semi-automatic, and automatic filling machines.

In some embodiments, the capsule having different sizes according to composition requirements. In some embodiments, the capsule size is: 000, 00, 0, 1, 2, 3, or 4. In some embodiments, the capsule size can be 000. In some embodiments, the capsule size can be 00. In some embodiments, the capsule size can be 0. In some embodiments, the capsule size can be 1. In some embodiments, the capsule size can be 2. In some embodiments, the capsule size can be 3. In some embodiments, the capsule size can be 4. In some embodiments, the capsule capacity varies from about 0.21 ml to about 1.37 ml. In some embodiments, the preferred capsule capacity is about 1.36 mL. In some embodiments, the preferred capsule dimensions are about 6.14 mm. length and about 9.91 mm external diameter. In some embodiments, the 000 capsule size is preferred. In some embodiments, the preferred capsule size is that needed to achieve the expanded gel volume of about 100 times to about 300 times the original volume of the capsule. In some embodiments, the preferred capsule size is that needed to achieve the expanded gel volume of about 200 times the original volume of the capsule. In some embodiments, the preferred capsule size is that needed to achieve the expanded gel volume of about 200 times the original volume of the capsule in acidic solution. In some embodiments, the preferred capsule size is that needed to achieve the expanded gel volume of about 100 milliliters to about 2,000 milliliters the original volume of the capsule. In some embodiments, the preferred capsule size is that needed to achieve the expanded gel volume of about 300 milliliters the original volume of the capsule. In some embodiments, the preferred capsule size is that needed to achieve the expanded gel volume of about 300 milliliters the original volume of the capsule in acidic solution. In some embodiments, the preferred capsule size is that needed to achieve the expanded gel volume of about 15-60% occupancy of the stomach. In some embodiments, the preferred capsule size is that needed to achieve the expanded gel volume of about 20-55% occupancy of the stomach.

In some embodiments, the composition described herein when stored in a sealed container placed in a room at 25° C. and a room atmosphere having about 50 percent relative humidity, retains at least about: 80%, 90%, 95%, 96%, 97%, 98%, or 99% of the active ingredient after 6 months, as measured by HPLC.

In some embodiments, the composition can be contained within a capsule, wherein the capsule can be loaded with about 25% to about 75% (by volume) with the composition. In some embodiments, the capsule can be loaded with about: 30%, 31%, 32%, 33%, 34%, 35%, 36%, 37%, 38%, 39% or about 40% (by volume) with a composition described herein. In some embodiments, the capsule can be loaded with about 25% to about 30%, about 25% to about 40%, about 25% to about 50%, about 25% to about 60%, about 25% to about 65%, about 25% to about 70%, about 25% to about 75%, about 30% to about 40%, about 30% to about 50%, about 30% to about 60%, about 30% to about 65%, about 30% to about 70%, about 30% to about 75%, about 40% to about 50%, about 40% to about 60%, about 40% to about 65%, about 40% to about 70%, about 40% to about 75%, about 50% to about 60%, about 50% to about 65%, about 50% to about 70%, about 50% to about 75%, about 60% to about 65%, about 60% to about 70%, about 60% to about 75%, about 65% to about 70%, about 65% to about 75%, or about 70% to about 75%, (by volume) with the composition.

In some embodiments, the content of the capsule comprises less than about: 10%, 9%, 8%, 7%, 6%, 5%, 4%, 3%, 2% or about 1% water by weight. In some embodiments, the content of the capsule comprises less than about 50%, about 40%, about 30%, about 25%, about 20%, about 10%, about 5%, or about 1% water by weight.

In some embodiments, the total content of all gases in the capsule can be less than about: 10%, 9%, 8%, 7%, 6%, 5%, 4%, 3%, 2% or about 1% water by weight. In some embodiments, the total content of all gases in the capsule can be less than about 50%, about 40%, about 30%, about 25%, about 20%, about 10%, about 5%, or about 1% water by weight.

In some embodiments, the capsule further comprises, in the volume not occupied by the composition, an inert gas. In some embodiments, the inert gas comprises an elemental gas, a compound gas, a noble gas, helium, neon, argon, krypton, xenon, radon, oganesson, compounds of noble gas, purified argon, purified nitrogen, nitrogen or any combination thereof. In some embodiments, the inert gas comprises nitrogen.

In some embodiments, administration can be by oral ingestion. In some embodiments, administration can comprise oral ingestion and the oral ingestion can comprise oral ingestion of a food, a liquid, a gel, a capsule, or any combination thereof. In some embodiments, In some embodiments, administration can be performed at least about: 1 time per day, 2 times per day, 3 times per day, 4 times per day, 5 times per day, 6 times per day or more than 6 times per day. In some embodiments, administration can be conducted one, twice, three, or four times per day. In some cases, administration can be provided by a subject (e.g. the patient), a health care provider, or both.

In some embodiments, the preferred dose of the composition(s) disclosed herein is that needed to achieve the expanded gel volume of about 100 times to about 300 times the original volume of the capsule. In some embodiments, the preferred dose is that needed to achieve the expanded gel volume of about 200 times the original volume of the capsule. In some embodiments, the preferred dose is that needed to achieve the expanded gel volume of about 200 times the original volume of the capsule in acidic solution. In some embodiments, the preferred dose is that needed to achieve the expanded gel volume of about 100 milliliters to about 2,000 milliliters the original volume of the capsule. In some embodiments, the preferred dose is that needed to achieve the expanded gel volume of about 300 milliliters the original volume of the capsule. In some embodiments, the preferred dose is that needed to achieve the expanded gel volume of about 300 milliliters the original volume of the capsule in acidic solution. In some embodiments, the preferred dose is that needed to achieve the expanded gel volume of about 15-60% occupancy of the stomach. In some embodiments, the preferred dose is that needed to achieve the expanded gel volume of about 20-55% occupancy of the stomach.

In some embodiments, multiple doses of the composition can be administered. In some embodiments, at least a single dose of the composition can be administered. In some embodiments, the composition can be administered so that a single dose of the active ingredients ranges from about 1.0 grams to about 25.0 grams of glucomannan (GMN), polysaccharide, magnesium metal powder (MMP), organic acid, excipients, or any combination thereof. In some embodiments, the composition can be administered so that a single dose of the active ingredients ranges from about 3.0 grams to about 10.0 grams of glucomannan (GMN), polysaccharide, magnesium metal powder (MMP), organic acid, excipients, or any combination thereof. In some embodiments, the composition can be administered so that a single dose of the active ingredients ranges from about 1.0 grams to about 25.0 grams of glucomannan (GMN), polysaccharide, magnesium metal powder (MMP), or any combination thereof. In some embodiments, the composition can be administered so that a single dose of the active ingredients ranges from about 3.0 grams to about 10.0 grams of glucomannan (GMN), polysaccharide, magnesium metal powder (MMP), or any combination thereof. In some embodiments, the composition can be administered so that a single dose of the active ingredients ranges from about 1.0 grams to about 25.0 grams of glucomannan (GMN), xanthan gum (XG), magnesium metal powder (MMP), or any combination thereof. In some embodiments, the composition can be administered so that a single dose of the active ingredients ranges from about 3.0 grams to about 10.0 grams of glucomannan (GMN), xanthan gum (XG), magnesium metal powder (MMP), or any combination thereof.

In some embodiments, the composition can be administered so that a single dose ranges from about 1.0 grams to about 25.0 grams of glucomannan (GMN). In some embodiments, the composition can be administered so that a single dose ranges from about 3.0 grams to about 10.0 grams of glucomannan (GMN). In some embodiments, the composition can be administered so that a single dose ranges from about 1 gram to about 25 grams of glucomannan (GMN). In some embodiments, the composition can be administered so that a single dose ranges from about about 1 gram to about 5 grams, about 1 gram to about 10 grams, about 1 gram to about 15 grams, about 1 gram to about 20 grams, about 1 gram to about 25 grams, about 5 grams to about 10 grams, about 5 grams to about 15 grams, about 5 grams to about 20 grams, about 5 grams to about 25 grams, about 10 grams to about 15 grams, about 10 grams to about 20 grams, about 10 grams to about 25 grams, about 15 grams to about 20 grams, about 15 grams to about 25 grams, or about 20 grams to about 25 grams of glucomannan (GMN). In some embodiments, the composition can be administered so that a single dose ranges from about 1 gram, about 5 grams, about 10 grams, about 15 grams, about 20 grams, or about 25 grams of glucomannan (GMN). In some embodiments, the composition can be administered so that a single dose ranges from about at least about 1 gram, about 5 grams, about 10 grams, about 15 grams, or about 20 grams of glucomannan (GMN). In some embodiments, the composition can be administered so that a single dose ranges from about at most about 5 grams, about 10 grams, about 15 grams, about 20 grams, or about 25 grams of glucomannan (GMN).

In some embodiments, the composition can be administered so that a single dose ranges from about 1.0 grams to about 25.0 grams of xanthan gum (XG). In some embodiments, the composition can be administered so that a single dose ranges from about 3.0 grams to about 10.0 grams of xanthan gum (XG). In some embodiments, the composition can be administered so that a single dose ranges from about 1 gram to about 25 grams of xanthan gum (XG). In some embodiments, the composition can be administered so that a single dose ranges from about 1 gram to about 5 grams, about 1 gram to about 10 grams, about 1 gram to about 15 grams, about 1 gram to about 20 grams, about 1 gram to about 25 grams, about 5 grams to about 10 grams, about 5 grams to about 15 grams, about 5 grams to about 20 grams, about 5 grams to about 25 grams, about 10 grams to about 15 grams, about 10 grams to about 20 grams, about 10 grams to about 25 grams, about 15 grams to about 20 grams, about 15 grams to about 25 grams, or about 20 grams to about 25 grams of xanthan gum (XG). In some embodiments, the composition can be administered so that a single dose ranges from about 1 gram, about 5 grams, about 10 grams, about 15 grams, about 20 grams, or about 25 grams of xanthan gum (XG). In some embodiments, the composition can be administered so that a single dose ranges from about at least about 1 gram, about 5 grams, about 10 grams, about 15 grams, or about 20 grams of xanthan gum (XG). In some embodiments, the composition can be administered so that a single dose ranges from about at most about 5 grams, about 10 grams, about 15 grams, about 20 grams, or about 25 grams of xanthan gum (XG).

In some embodiments, the composition can be administered so that a single dose ranges from about 1.0 grams to about 25.0 grams of magnesium metal powder (MMP). In some embodiments, the composition can be administered so that a single dose ranges from about 3.0 grams to about 10.0 grams of magnesium metal powder (MMP). In some embodiments, the composition can be administered so that a single dose ranges from about 1 gram to about 25 grams of magnesium metal powder (MMP). In some embodiments, the composition can be administered so that a single dose ranges from about 1 gram to about 5 grams, about 1 gram to about 10 grams, about 1 gram to about 15 grams, about 1 gram to about 20 grams, about 1 gram to about 25 grams, about 5 grams to about 10 grams, about 5 grams to about 15 grams, about 5 grams to about 20 grams, about 5 grams to about 25 grams, about 10 grams to about 15 grams, about 10 grams to about 20 grams, about 10 grams to about 25 grams, about 15 grams to about 20 grams, about 15 grams to about 25 grams, or about 20 grams to about 25 grams of magnesium metal powder (MMP). In some embodiments, the composition can be administered so that a single dose ranges from about 1 gram, about 5 grams, about 10 grams, about 15 grams, about 20 grams, or about 25 grams of magnesium metal powder (MMP). In some embodiments, the composition can be administered so that a single dose ranges from about at least about 1 gram, about 5 grams, about 10 grains, about 15 grams, or about 20 grams of magnesium metal powder (MMP). In some embodiments, the composition can be administered so that a single dose ranges from about at most about 5 grams, about 10 grams, about 15 grams, about 20 grams, or about 25 grams of magnesium metal powder (MMP).

In some embodiments, administration can be performed for about: 1 day to about 8 days, 1 week to about 5 weeks, 1 month to about 12 months, 1 year to about 3 years, 3 years to about 10 years, 10 years to about 50 years, 25 years to about 100 years, or about 50 years to about 130 years.

b. Co-Administration

In some embodiments, a method can further comprise co-administration of the composition and food to the subject. In some embodiments, the co-administration of the composition and food comprises administration of the composition from about 0 minutes to about 50 minutes after the administration of food. In some embodiments, the composition can be administered so that the time between the administration of food and the administration of the composition ranges from about 0 minutes to about 50 minutes. In some embodiments, the composition can be administered so that the time between the administration of food and the administration of the composition ranges from about 0 minutes to about 5 minutes, about 0 minutes to about 10 minutes, about 0 minutes to about 15 minutes, about 0 minutes to about 20 minutes, about 0 minutes to about 25 minutes, about 0 minutes to about 30 minutes, about 0 minutes to about 35 minutes, about 0 minutes to about 40 minutes, about 0 minutes to about 45 minutes, about 0 minutes to about 50 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 35 minutes, about 5 minutes to about 40 minutes, about 5 minutes to about 45 minutes, about 5 minutes to about 50 minutes, about 10 minutes to about 15 minutes, about 10 minutes to about 20 minutes, about 10 minutes to about 25 minutes, about 10 minutes to about 30 minutes, about 10 minutes to about 35 minutes, about 10 minutes to about 40 minutes, about 10 minutes to about 45 minutes, about 10 minutes to about 50 minutes, about 15 minutes to about 20 minutes, about 15 minutes to about 25 minutes, about 15 minutes to about 30 minutes, about 15 minutes to about 35 minutes, about 15 minutes to about 40 minutes, about 15 minutes to about 45 minutes, about 15 minutes to about 50 minutes, about 20 minutes to about 25 minutes, about 20 minutes to about 30 minutes, about 20 minutes to about 35 minutes, about 20 minutes to about 40 minutes, about 20 minutes to about 45 minutes, about 20 minutes to about 50 minutes, about 25 minutes to about 30 minutes, about 25 minutes to about 35 minutes, about 25 minutes to about 40 minutes, about 25 minutes to about 45 minutes, about 25 minutes to about 50 minutes, about 30 minutes to about 35 minutes, about 30 minutes to about 40 minutes, about 30 minutes to about 45 minutes, about 30 minutes to about 50 minutes, about 35 minutes to about 40 minutes, about 35 minutes to about 45 minutes, about 35 minutes to about 50 minutes, about 40 minutes to about 45 minutes, about 40 minutes to about 50 minutes, or about 45 minutes to about 50 minutes. In some embodiments, the composition can be administered so that the time between the administration of food and the administration of the composition ranges from about 0 minutes, about 5 minutes, about 10 minutes, about 15 minutes, about 20 minutes, about 25 minutes, about 30 minutes, about 35 minutes, about 40 minutes, about 45 minutes, or about 50 minutes. In some embodiments, the composition can be administered so that the time between the administration of food and the administration of the composition ranges from about at least about 0 minutes, about 5 minutes, about 10 minutes, about 15 minutes, about 20 minutes, about 25 minutes, about 30 minutes, about 35 minutes, about 40 minutes, or about 45 minutes. In some embodiments, the composition can be administered so that the time between the administration of food and the administration of the composition ranges from about at most about 5 minutes, about 10 minutes, about 15 minutes, about 20 minutes, about 25 minutes, about 30 minutes, about 35 minutes, about 40 minutes, about 45 minutes, or about 50 minutes.

In some embodiments, the composition can be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some embodiments, the composition in a capsule, the composition as a hydrogel, and the powder composition can be co-administered with food. In some embodiments, the composition in a capsule, and the composition as a hydrogel can be co-administered with food. In some embodiments, the composition in a capsule, and the powder composition can be co-administered with food. In some embodiments, the composition as a hydrogel, and the powder composition can be co-administered with food. In some embodiments, the composition can be co-administered with food. In some embodiments, the composition in a capsule can be co-administered with food. In some embodiments, the composition as a hydrogel can be co-administered with food. In some embodiments, the powder composition can be co-administered with food. In some embodiments, multiple doses of the composition can be co-administered with food. In some embodiments, at least a single dose of the composition can be co-administered with food.

In some embodiments, the composition can be co-administered so that a single dose of the active ingredients ranges from about 1.0 grams to about 25.0 grams of glucomannan (GMN), polysaccharide, magnesium metal powder (MMP), organic acid, excipients, or any combination thereof. In some embodiments, the composition can be co-administered so that a single dose of the active ingredients ranges from about 3.0 grams to about 10.0 grams of glucomannan (GMN), polysaccharide, magnesium metal powder (MMP), organic acid, excipients, or any combination thereof. In some embodiments, the composition can be co-administered so that a single dose of the active ingredients ranges from about 1.0 grams to about 25.0 grams of glucomannan (GMN), polysaccharide, magnesium metal powder (MMP), or any combination thereof. In some embodiments, the composition can be co-administered so that a single dose of the active ingredients ranges from about 3.0 grams to about 10.0 grams of glucomannan (GMN), polysaccharide, magnesium metal powder (MMP), or any combination thereof. In some embodiments, the composition can be co-administered so that a single dose of the active ingredients ranges from about 1.0 grams to about 25.0 grams of glucomannan (GMN), xanthan gum (XG), magnesium metal powder (MMP), or any combination thereof. In some embodiments, the composition can be co-administered so that a single dose of the active ingredients ranges from about 3.0 grams to about 10.0 grams of glucomannan (GMN), xanthan gum (XG), magnesium metal powder (MMP), or any combination thereof.

In some embodiments, the composition can be co-administered so that a single dose ranges from about 1.0 grams to about 25.0 grams of glucomannan (GMN). In some embodiments, the composition can be co-administered so that a single dose ranges from about 3.0 grams to about 10.0 grams of glucomannan (GMN). In some embodiments, the composition can be co-administered so that a single dose ranges from about 1 gram to about 25 grams of glucomannan (GMN). In some embodiments, the composition can be co-administered so that a single dose ranges from about 1 gram to about 5 grams, about 1 gram to about 10 grams, about 1 gram to about 15 grams, about 1 gram to about 20 grams, about 1 gram to about 25 grams, about 5 grams to about 10 grams, about 5 grams to about 15 grams, about 5 grams to about 20 grams, about 5 grams to about 25 grams, about 10 grams to about 15 grams, about 10 grams to about 20 grams, about 10 grams to about 25 grams, about 15 grams to about 20 grams, about 15 grams to about 25 grams, or about 20 grams to about 25 grams of glucomannan (GMN). In some embodiments, the composition can be co-administered so that a single dose ranges from about 1 gram, about 5 grams, about 10 grams, about 15 grams, about 20 grams, or about 25 grams of glucomannan (GMN). In some embodiments, the composition can be co-administered so that a single dose ranges from about at least about 1 gram, about 5 grams, about 10 grams, about 15 grams, or about 20 grams of glucomannan (GMN). In some embodiments, the composition can be co-administered so that a single dose ranges from about at most about 5 grams, about 10 grams, about 15 grams, about 20 grams, or about 25 grams of glucomannan (GMN).

In some embodiments, the composition can be co-administered so that a single dose ranges from about 1.0 grams to about 25.0 grams of xanthan gum (XG). In some embodiments, the composition can be co-administered so that a single dose ranges from about 3.0 grams to about 10.0 grams of xanthan gum (XG). In some embodiments, the composition can be co-administered so that a single dose ranges from about 1 gram to about 25 grams of xanthan gum (XG). In some embodiments, the composition can be co-administered so that a single dose ranges from about 1 gram to about 5 grams, about 1 gram to about 10 grams, about 1 gram to about 15 grams, about 1 gram to about 20 grams, about 1 gram to about 25 grams, about 5 grams to about 10 grams, about 5 grams to about 15 grams, about 5 grams to about 20 grams, about 5 grams to about 25 grams, about 10 grams to about 15 grams, about 10 grams to about 20 grams, about 10 grams to about 25 grams, about 15 grams to about 20 grams, about 15 grams to about 25 grams, or about 20 grams to about 25 grams of xanthan gum (XG). In some embodiments, the composition can be co-administered so that a single dose ranges from about 1 gram, about 5 grams, about 10 grams, about 15 grams, about 20 grams, or about 25 grams of xanthan gum (XG). In some embodiments, the composition can be co-administered so that a single dose ranges from about at least about 1 gram, about 5 grams, about 10 grams, about 15 grams, or about 20 grams of xanthan gum (XG). In some embodiments, the composition can be co-administered so that a single dose ranges from about at most about 5 grams, about 10 grams, about 15 grams, about 20 grams, or about 25 grams of xanthan gum (XG).

In some embodiments, the composition can be co-administered so that a single dose ranges from about 1.0 grams to about 25.0 grams of magnesium metal powder (MMP). In some embodiments, the composition can be co-administered so that a single dose ranges from about 3.0 grams to about 10.0 grams of magnesium metal powder (MMP). In some embodiments, the composition can be co-administered so that a single dose ranges from about 1 gram to about 25 grams of magnesium metal powder (MMP). In some embodiments, the composition can be co-administered so that a single dose ranges from about 1 gram to about 5 grams, about 1 gram to about 10 grams, about 1 gram to about 15 grams, about 1 gram to about 20 grams, about 1 gram to about 25 grams, about 5 grams to about 10 grams, about 5 grams to about 15 grams, about 5 grams to about 20 grams, about 5 grams to about 25 grams, about 10 grams to about 15 grams, about 10 grams to about 20 grams, about 10 grams to about 25 grams, about 15 grams to about 20 grams, about 15 grams to about 25 grams, or about 20 grams to about 25 grams of magnesium metal powder (MMP). In some embodiments, the composition can be co-administered so that a single dose ranges from about 1 gram, about 5 grams, about 10 grams, about 15 grams, about 20 grams, or about 25 grams of magnesium metal powder (MMP). In some embodiments, the composition can be co-administered so that a single dose ranges from about at least about 1 gram, about 5 grams, about 10 grams, about 15 grams, or about 20 grams of magnesium metal powder (MMP). In some embodiments, the composition can be co-administered so that a single dose ranges from about at most about 5 grams, about 10 grams, about 15 grams, about 20 grams, or about 25 grams of magnesium metal powder (MMP).

c. Methods of Treating or Preventing a Disease

Also disclosed herein are methods of treating or preventing a disease comprising treating or preventing the disease or condition by administering a therapeutically effective amount of the composition or compositions. Also disclosed herein are methods of treating or preventing a disease comprising treating or preventing the disease or condition by administering, via oral administration, a therapeutically effective amount of the composition or compositions. In some embodiments, the disease can comprise treating or preventing a disease or condition selected from the group consisting of: hypertension, pulmonary arterial hypertension, heart disease, arrhythmia, cardiomyopathy, high blood pressure, high cholesterol, irregular levels of triglycerides, prediabetes, Type 2 diabetes, coronary heart disease, gallbladder disease, osteoarthritis, obesity, gout, sleep apnea, stroke, metabolic syndrome, cancer, gastroesophageal reflux, kidney disease, liver disease, joint diseases, weight gain, chronic exhaustion, inflammatory diseases, or any combination thereof. In some embodiments, administration of the compositions disclosed herein comprises treating or preventing a disease or condition selected from the group consisting of: hypertension, pulmonary arterial hypertension, heart disease, arrhythmia, cardiomyopathy, high blood pressure, high cholesterol, irregular levels of triglycerides, prediabetes, Type 2 diabetes, coronary heart disease, gallbladder disease, osteoarthritis, obesity, gout, sleep apnea, stroke, metabolic syndrome, cancer, gastroesophageal reflux, kidney disease, liver disease, joint diseases, weight gain, chronic exhaustion, inflammatory diseases, or any combination thereof. In some embodiments, administration of at least one of the compositions disclosed herein comprises treating or preventing a disease or condition selected from the group as previously disclosed. In some embodiments, administration of at least one of the compositions disclosed herein comprises treating or preventing a disease or condition from the group consisting of metabolic disease, weight gain, chronic exhaustion, inflammatory diseases, or any combination thereof.

In some embodiments, prior to treating, a subject may have been diagnosed with the disease. In some embodiments, the subject may be a human, a man, a woman, an individual over 18 years of age, an individual under 18 years of age, or any combination thereof.

In some embodiments, a subject can be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

In some embodiments, a method can further comprise diagnosing a subject as having the disease. In some embodiments, a diagnosing can comprise employing an in vitro diagnostic. In some embodiments, the in vitro diagnostic can be a companion diagnostic.

In some embodiments, a diagnosis can comprise a physical examination, a radiological image, a blood test, an antibody test, or any combination thereof. In some embodiments, a diagnosis can comprise a radiological image and the radiological image can comprise: a computed tomography (CT) image, an X-Ray image, a magnetic resonance image (MRI), an ultrasound image, or any combination thereof.

In some embodiments, a method can further comprise administering a second therapy or composition to the subject. In some embodiments a method can further comprise administering a second therapy or composition to the subject wherein the second therapy or composition is one of the compositions disclosed herein. In some embodiments a method can further comprise administering a second therapy or composition to the subject wherein the second therapy or composition is the powder composition, the composition in a capsule, the composition as a hydrogel, or any combination thereof. In some embodiments, a second therapy or composition can comprise acetaminophen, an opioid, a nonsteroidal anti-inflammatory drug, methotrexate, hydroxychloroquine, prednisone, cortisone, a biological response modifier, a salt thereof, or any combination thereof. In some embodiments, a second therapy or composition can comprise a biological response modifier and the biological response modifier can comprise: abatacept, adalimumab, adalimumab-atto, anakinra, certolizumab pegol, etanercept, etanercept-szzs, golimumab, infliximab, infliximab-dyyb, rituximab, sarilumab, tocilizumab, a biologically active fragment of any of these, a salt of any of these, or any combination thereof. In some embodiments, the second therapy or composition can comprise a nonsteroidal anti-inflammatory drug and the nonsteroidal anti-inflammatory drug can comprise naproxen, ibuprofen, a salt of any of these, or any combination thereof. In some embodiments, a composition can comprise an excipient, a diluent, a carrier, or any combination thereof.

In some embodiments, the second therapy or composition can be administered as needed, or for: one day, two days, three days, four days, five days, six days, a week, two weeks, three weeks, a month, two months, three months, four months, five months, six months, seven months, eight months, nine months, ten months, eleven months, a year, or chronically.

### XI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

In the description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the terms “determining,” “measuring,” “evaluating,” “assessing,” “assaying,” and “analyzing” are often used interchangeably herein to refer to forms of measurement. As used herein the terms include determining if an element is present or not (for example, detection). As used herein these terms can include quantitative, qualitative or quantitative and qualitative determinations. Assessing can be relative or absolute. “Detecting the presence of” can include determining the amount of something present in addition to determining whether it is present or absent depending on the context.

As used herein the terms “subject,” “individual,” or “patient” are often used interchangeably herein. As used herein the “subject” can be a biological entity containing expressed genetic materials. The biological entity can be a plant, animal, or microorganism, including, for example, bacteria, viruses, fungi, and protozoa. As used herein the subject or individual can be tissues, cells and their progeny of a biological entity obtained in vivo or cultured in vitro. As used herein the term “individual” refers to live organisms that are human or Homo sapiens species, or not human or not Homo sapiens species. In certain embodiments, the individual is a human. In certain embodiments, the individual is a mammal. In certain embodiments, the mammal is a human, mouse, rat, rabbit, dog, cat, horse, cow, sheep, pig, goat, cattle, boar, or deer. In certain embodiments, the non-human animal is selected from the group consisting of: a cow, a pig, a chicken, a fish, a bird, a sheep, a bison, a cattle, a boar, a sheep, a goat, a duck, and a turkey. In certain embodiments, the individual may be diagnosed or suspected of being at high risk for a disease. In certain embodiments, the individual is not necessarily diagnosed or suspected of being at high risk for the disease.

As used herein the term “in vivo” is used to describe an event that takes place in a subject's body.

As used herein the term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

As used herein the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. Compositions for treating or preventing a given disease can consist essentially of the recited active ingredient, exclude additional active ingredients, but include other non-material components such as excipients, carriers, or diluents. “Consisting of” shall mean excluding more than trace elements of other ingredients and substantial method steps. Embodiments defined by each of these transition terms are within the scope of this disclosure.

As used herein the term “about” refers to an amount that is near the stated amount by plus or minus 10%. As used herein the term “about” a range refers to that range minus 10% of its lowest value and plus 10% of its greatest value.

As used herein the term “a.u.” or “arbitrary unit” refers to a relative unit of measurement to show the ratio of amount of quantities or proportions, and serves to compare multiple measurements performed in similar environment since the ratio between measurement and reference is consistent and dimensionless quantity independent of what actual units are used. In certain embodiments, the ratio is a percentage of the proportions of the control sample.

As used herein the term “ppm” or “parts per million” refers to a unit of measurement to show the number of units of mass, or parts, per million units of total mass. As used herein the term “ppm” refers to a relative unit of measurement to show the ratio of amount of quantities or proportions, and serves to compare multiple measurements performed in similar environment since the ratio between measurement and reference is consistent and dimensionless quantity independent of what actual units are used. In certain embodiments, the ratio is a percentage of the proportions of the control sample.

As used herein the term “co-administration” refers to the simultaneous intake of food and non-food compositions or formulations wherein the introduction of food and non-food compositions or formulations into the stomach of an individual occurs within a window of time ranging from about 30 minutes to about 60 minutes. As used herein the term “co-administration” refers to a relative window of time wherein the food and non-food compositions or formulations are both in the stomach of an individual, meaning the gastric phase of the food and non-food compositions or formulations overlaps, stimulating gastric activity at the same time. As used herein the term “co-administration” refers to simultaneous intake of food and non-food compositions or formulations, wherein co-administration can modify release, absorption, distribution, metabolism and/or elimination and consequently, the non-food compositions or formulations following oral drug administration.

As used herein the term “encapsulated” or “encapsulation” refers to a state of being enclosed, contained, or surrounded by a capsule. As used herein the term “encapsulated” refers to a state of being within a capsule. As used herein the term “encapsulated” refers to a state of being contained within a gelatinous or membranous envelope. As used herein the term “encapsulation” refers to containing materials such as drugs, vitamins and supplements in enclosed compartments commonly referred to capsules.

As used herein the term “floating” refers to a state of being buoyant or suspended in liquid. As used herein the term “floating” refers to a state of not being anchored in a particular place or position within a volume.

As used herein the term “expand” or “expansive” refers to tending or being able to grow in size, volume, or area. As used herein the term “expand” refers to tending or being able to get larger or filling more space. As used herein the term “expand” refers to tending or being able to become more extensive. As used herein the term “expand” refers to tending or being able to dilate, distend, inflate spread out, usually in every direction.

As used herein the term “hydrogel” refers to a cross-linked network of monomer units, or a polymer structure comprising a plurality of molecules that make up at least a portion of the polymer structure. A plurality of monomers can attach to one another in order to form a polymer chain, a branched polymer, a crosslinked polymer, and the like. In some embodiments, the hydrogel forms after the monomers undergo polymerization to form a polymer structure. As used herein, the term “polymer structure” can refer to an ordered structure of molecules.

As used herein the term “satiety” refers to tending to be in the quality or state of being fed or gratified to or beyond capacity. As used herein the term “satiety” refers to the absence of hunger. As used herein the term “satiety” refers to the sense of fullness after eating.

As used herein the term “weight loss” refers to a reduction of the total body mass or a decrease in body weight. As used herein, the term “weight loss” refers to intentional or unintentional decrease in body weight, either with or without a change in appearance. Weight loss or a reduction of the total body mass can occur wherein body mass is comprised of different components such as, for example, fluid, body fat (adipose tissue), or lean mass (such as bone mineral deposits, muscle, tendon, connective tissue, or other combinations thereof).

As used herein the term “exercise endurance” refers to improving exercise tolerance or enhancing exercise performance in an individual or subject. As used herein “exercise endurance” refers to the ability of an organism to exert itself and remain active for an extended period of time, as well as its ability to resist, withstand, recover from, and have immunity to trauma, wounds, or fatigue. As used herein “exercise” refers to both aerobic or anaerobic exercise.

As used herein the term “anti-inflammatory” or “anti-inflammatory effects” refers to the property of a substance or treatment that reduces inflammation or swelling in the body, counteracting inflammation. As used herein the term “anti-inflammatory” refers to the effect of preventing or reducing inflammation in the body, such as, for example redness, tenderness, swelling, tissue or cellular swelling, liquid retention, and pain. Anti-inflammatory substances or treatment agents can block certain substances in the body that cause inflammation, or upregulate pathways that reduce inflammation.

As used herein the term “pouch” or “packet” refers to a small compartment, contained, or envelope that encloses or contains a material, product, or composition. In some instances, packets provide small or single-use amounts of a material, product, or composition. In some instance, packets allow for ease of availability or distribution small or single-use amounts of a material, product, or composition.

As used herein the term “molecular hydrogen” refers to a molecule formed by two hydrogen atoms that share their electrons. As used herein the term “molecular hydrogen” or “dihydrogen” refers to a diatomic molecule that is composed of two hydrogen atoms held together by a covalent bond.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “probiotic” means live microorganisms intended to provide health benefits when consumed, generally by improving or restoring the gut flora.

The term “prebiotic” means compounds in food that induce the growth or activity of beneficial microorganisms such as bacteria and fungi.

The term “sustainability” is defined herein as the ability to hold H₂ in gels or solutions over the short-term.

The term “stability” is defined as the ability to hold H₂ in a gel over the longer—term, i.e., longer than 24 hours.

### DETAILED DESCRIPTION OF THE DRAWINGS

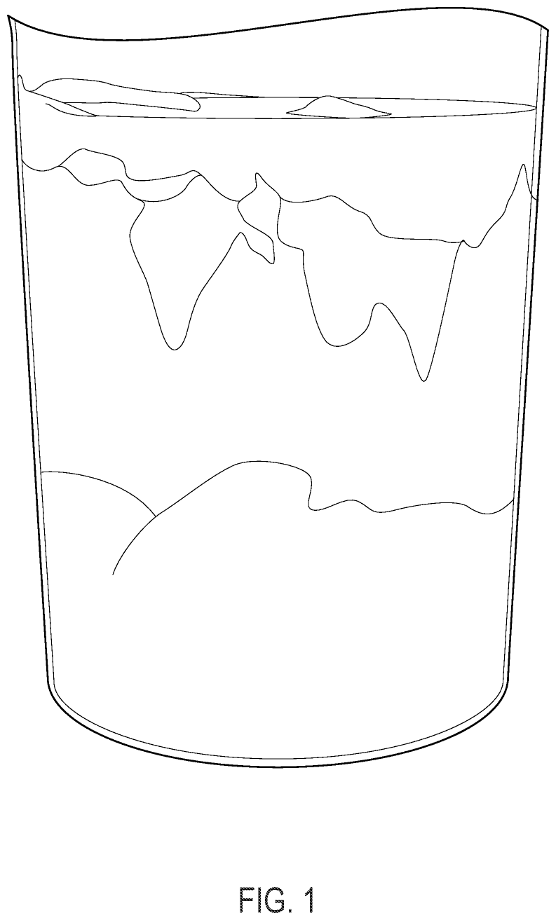

FIG. 1 depicts an image of three size 000 capsules containing 1:1 glucomannan:xanthan gum dissolved in 300 mL of 5% acetic acid for 120-minutes in a Griffin beaker flask with flat bottom, according to at least some embodiments disclosed herein. FIG. 1 demonstrates a lack of volume expansion and a phase separation of gel when three-000 capsules containing a total of 2.4 grams of 1:1 glucomannan:xanthan gum are mixed with 5% acetic acid in absence of magnesium metal powder.

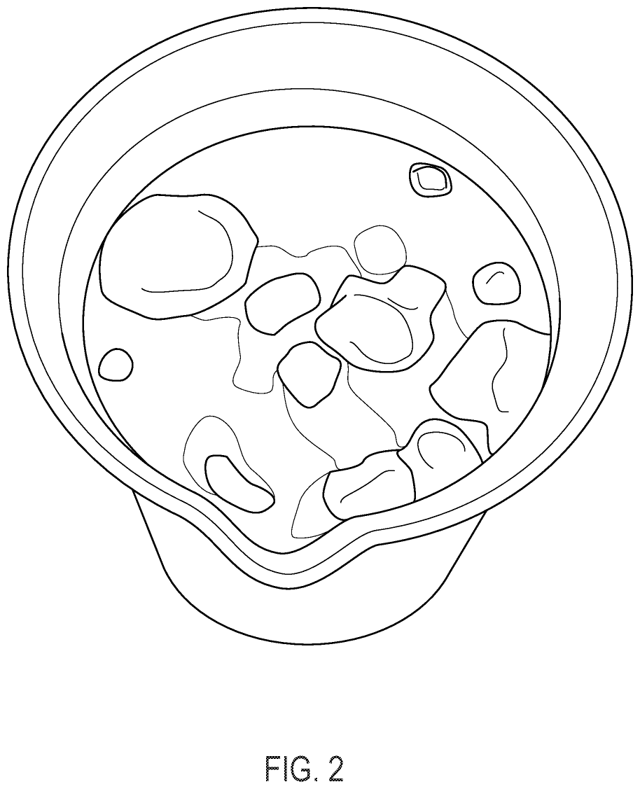

FIG. 2 depicts an image of the top view of the capsular contents of the same preparation depicted in FIG. 1, according to at least some embodiments disclosed herein. FIG. 2 demonstrates the clumps/blobs/dense groupings of gel resulting from non-expanded capsules and capsule contents.

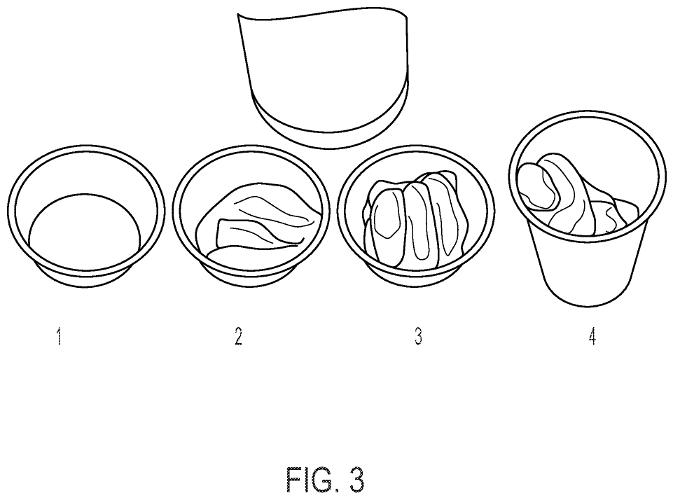

FIG. 3 depicts an image of four capsules containing glucomannan (NOW® Foods) in aqueous-acidic solution, according to at least some embodiments disclosed herein. FIG. 3 demonstrated that capsules containing glucomannan dissolved in aqueous-acidic solution form a non-expanded dense grouping of gel.

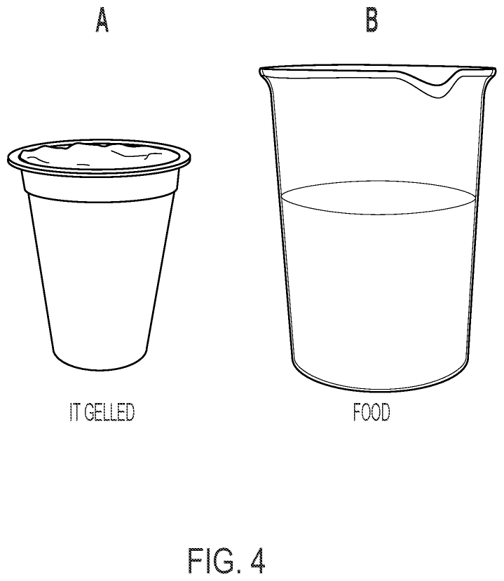

FIG. 4 depicts an image of (A) three capsules containing 1T (see Table 3) dissolved in 350 mL of a vinegar solution in a cup, (B) 99.7 grams of a simulated food sample dissolved in 650 mL of 5% acetic acid in a cup, according to at least some embodiments disclosed herein. FIG. 4 depicts the individual components prior to co-administration, when individual components are composition comprised in capsule and food.

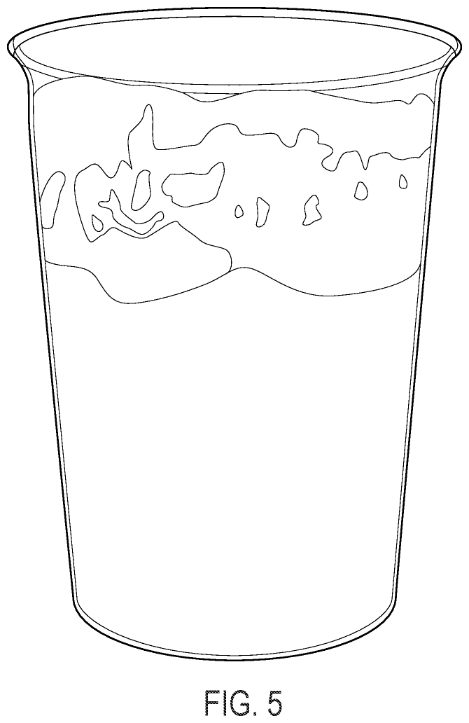

FIG. 5 depicts an image of the contents of the same preparations depicted in FIGS. 4A and 4B in a Griffin beaker flask prior to mixing, according to at least some embodiments disclosed herein. FIG. 5 demonstrates the effects on the co-administration of food and compositions disclosed herein. In particular, FIG. 5 shows food and three capsules of 1T dissolved in vinegar solution, or acidic solution shortly after co-administration (prior to mixing). FIG. 5 depicts the gel floats to the top of the volume within the beaker.

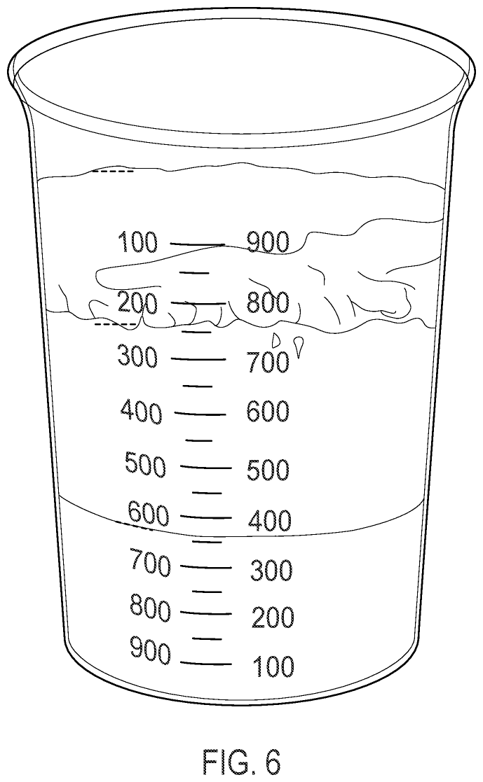

FIG. 6 depicts an image of the contents of the same preparations depicted in FIGS. 4A and 4B in a Griffin beaker flask after mixing, according to at least some embodiments disclosed herein. FIG. 6 demonstrates the effects on the co-administration of food and compositions disclosed herein. In particular, FIG. 6 shows food and three capsules of 1T dissolved in vinegar solution, or acidic solution after co-administration and mixing. FIG. 6 depicts the mixture following co-administration comprises three distinct phases, or three distinct densities in the volume within the beaker.

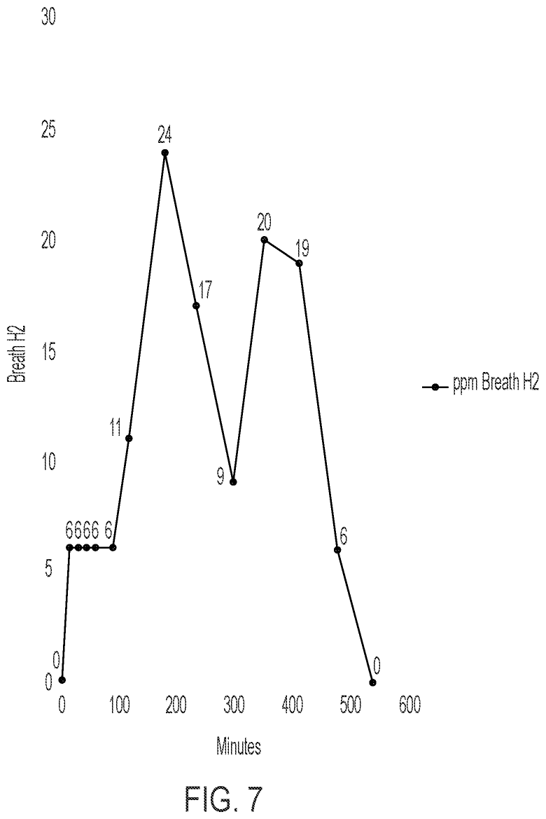

FIG. 7 depicts a graph of molecular hydrogen measured in breath of individual (as parts per million) after co-administration of food and three capsules containing 1T as a function of time (in minutes), according to at least some embodiments disclosed herein. FIG. 7 depicts that the resultant food bolus may reside in the stomach of an individual for as long as 8-hours.

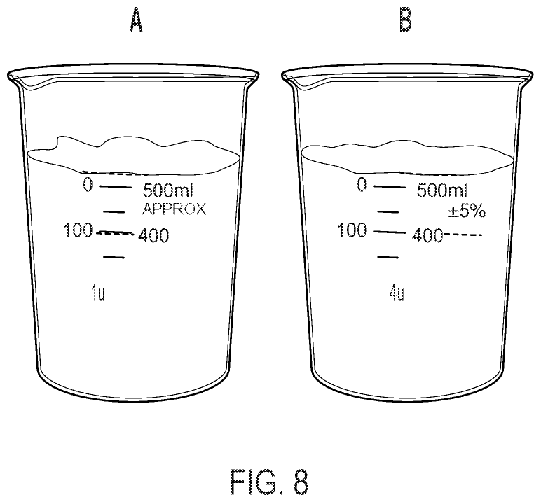

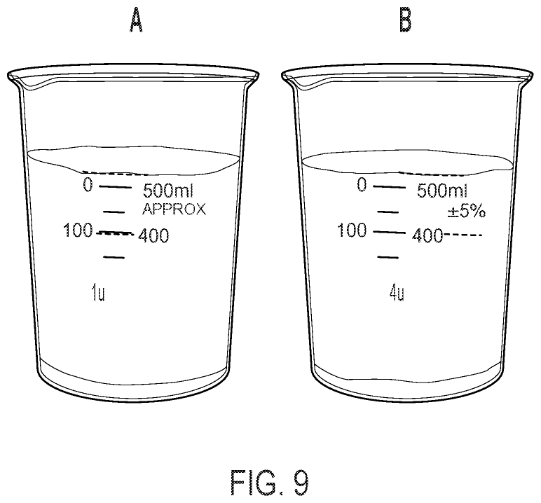

FIG. 8 depicts an image of (A) three size 000 capsules containing 1:1.2 glucomannan:xanthan gum+0.667 grams of magnesium metal powder (MMP) (Capsule 1U; see Table 4) dissolved in 100 mL of 5% acetic acid 60 minutes after mixing in a Griffin beaker flask with flat bottom, (B) three size 000 capsules containing 1:1.2 glucomannan:xanthan gum+1.043 grams of magnesium metal powder (Capsule 4U; see Table 4) dissolved in 100 mL of 5% acetic acid 60 minutes after mixing in a Griffin beaker flask with flat bottom, according to at least some embodiments disclosed herein. FIG. 9 depicts a visual of the volume expansion of the gel in the presence of magnesium metal powder. As shown in FIG. 9, the gel comprising a higher concentration of magnesium metal powder (4U) expands at a greater volume as compared to the gel comprising a lower concentration of magnesium metal powder (1U).

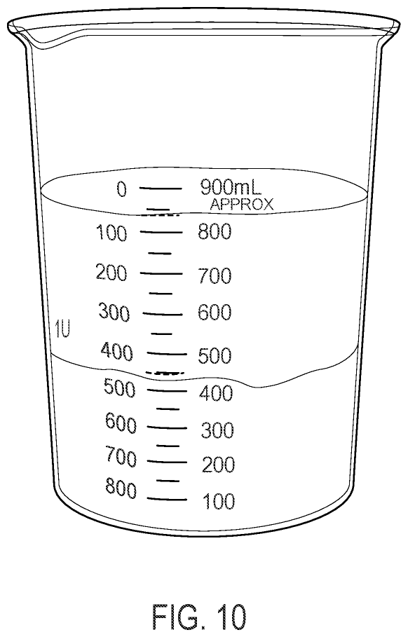

FIG. 9 depicts an image of the same preparations depicted in FIG. 9, about 6 hours post-mixing, according to at least some embodiments disclosed herein. FIG. 10 shows that the gel volume expansion depicted in FIG. 9 is sustained after being allowed to stand overnight.

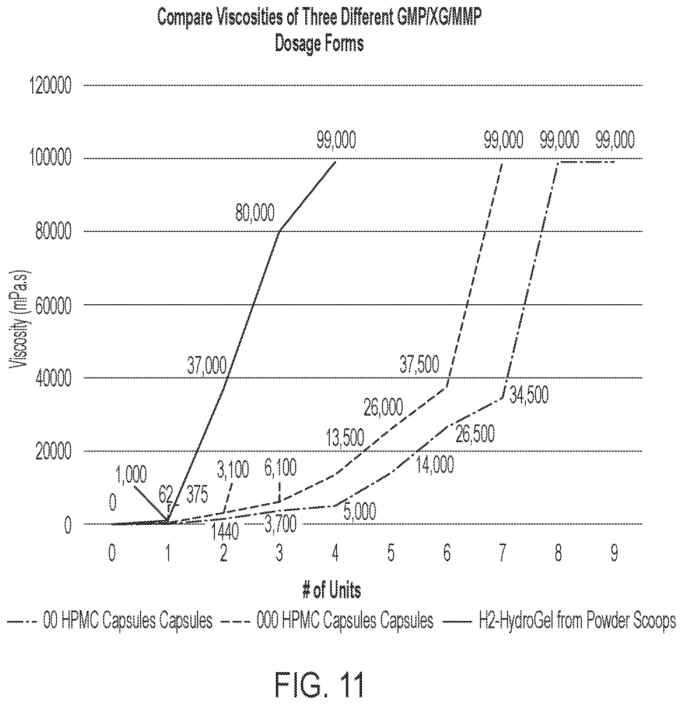

FIG. 10 depicts an image of a hydrogel created after mixing two size 000 capsules of 1U (Table 4) with 400 mL of vinegar solution after addition of another 400 mL of vinegar solution in a Griffin beaker flask with flat bottom, according to at least some embodiments disclosed herein. FIG. 11 shows the volume of the floating expanded gel created after mixing two size 000 capsules of 1U with 400 mL of vinegar solution is about 425 mL.

FIG. 11. depicts a graph of viscosity for three GMP/XG/MMP dosages (00 HPMC capsule, 000 HPMC capsule, and H2-Hydrogel from powder scoop) measured in mPa·s as a function of number of units. All three dosage forms display an exponential increase in viscosity as the number of units increased.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention

### Specific Embodiments

Certain specific embodiments are envisioned which include:

1. A method of administrating a composition capable of generating molecular hydrogen to an individual, the method comprising: co-administering food and a dose or doses of the composition to the alimentary canal of the individual, wherein the composition comprises glucomannan, xanthan gum, magnesium metal powder, or any combination thereof.

2. The method of claim 1, wherein the composition is enclosed in a capsule.

3. The method of claim 1, wherein the composition is formulated for oral delivery.

4. The method of claim 1, wherein the composition is formulated to induce satiety/weight loss.

5. The method of claim 1, wherein the composition is formulated to induce exercise endurance.

6. The method of claim 1, wherein the composition is formulated to induce anti-inflammatory effects.

7. The method of claim 1, wherein co-administration of food and a dose of the composition comprises releasing the composition into the stomach of the individual at most about 40 minutes following the introduction of food into the stomach of the individual.

8. The method of any one of claims 1 to 7, wherein the composition comprises at least about 2 wt % to at least about 98 wt % of glucomannan.

9. The method of any one of claims 1 to 7, wherein the composition comprises from about 40 wt % to about 50 wt % of glucomannan.

10. The method of any one of claims 1 to 7, wherein the composition comprises at least about 0.0 wt % to at least about 98 wt % of xanthan gum.

11. The method of any one of claims 1 to 7, wherein the composition comprises from about 40 wt % to about 50 wt % of xanthan gum.

12. The method of any one of claims 1 to 7, wherein the composition comprises at least about 0.001 wt % to at least about 30 wt % of magnesium metal powder.

13. The method of any one of claims 1 to 7, wherein the composition comprises from about 0.001 wt % to about 5 wt % of magnesium metal powder.

14. The method of any one of claims 1 to 13, wherein the composition is released from the capsule following co-administration.

15. The method of claim 1, wherein the composition is enclosed in the capsule that is at least 0, 00, or 000 in size.

16. The method of claim 1, wherein the capsule comprises cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate, hydroxypropyl methylcellulose (HPMC), gelatin, polysaccharide, or any combination thereof.

17. The method of claim 1, wherein the capsule is made from hydroxypropyl methylcellulose (HPMC), cellulose, gelatin, or any combination thereof.

18. The method of claim 1, wherein the method comprises co-administration of food and at least 2 capsules comprising a dose of the composition.

19. The method of claim 1, wherein the method comprises co-administration of food and at least 3 capsules comprising a dose of the composition.

20. The method of claim 1, wherein following co-administration of food and at least one capsule comprising a dose of the composition, the composition expands to a volume of about 100 milliliters to about 2,000 milliliters in acidic solution to form a hydrogel.

21. The method of claim 20, wherein the composition expands to a volume of about 300 milliliters.

22. The method of claim 1, wherein following co-administration of food and at least one capsule comprising a dose of the composition, the composition associates with acidic solution of the individual's stomach to form a hydrogel that expands to a volume of about 100 times to about 200 times the original volume of the capsule.

23. The method of claim 22, wherein the composition associates with acidic solution of the individual's stomach to form a hydrogel that expands to a volume of about 300 times the original volume of the capsule.

24. The method of claim 1, wherein following co-administration of food and at least one capsule comprising a dose of the composition, the hydrogel comprises a volume equivalent to about 15% to about 60% of the individual's stomach capacity.

25. The method of any one of claims 20 to 24, wherein the hydrogel comprises a volume sufficient to induce satiety in the individual.

26. The method of any one of claims 20 to 24, wherein the hydrogel comprises a volume sufficient to induce weight loss in the individual.

27. The method of any one of claims 20 to 24, wherein the hydrogel remains at a constant volume within the stomach of the individual for at least about 4 hours to at least about 7 hours.

28. The method of any one of claims 20 to 24, wherein the composition expands in acidic solution to form a hydrogel, generating molecular hydrogen by reaction of magnesium metal powder with the acidic solution.

29. The method of any one of claims 20 to 24, wherein the molecular hydrogen accelerates the dissolution of the capsule containing the composition in the acidic solution.

30. The method of any one of claims 20 to 24, wherein the molecular hydrogen induces satiety/weight loss, exercise endurance, anti-inflammatory effects, or any combination thereof.

31. The method of any one of claims 20 to 24, wherein the acidic solution is stomach acid.

32. The method of claim 30, wherein satiety or weight loss is induced following co-administration of food and at least one capsule comprising a dose of the composition when ingested at least once a day.

33. The method of claim 32, wherein daily co-administration of food and at least one capsule comprising a dose of the composition reduces body weight, induces exercise endurance, and/or induces anti-inflammatory effects in the individual over an extended period of time.

34. The method of claim 33, wherein an extended period of time comprises at least about 1 week.

35. The method of claim 33, wherein an extended period of time comprises about 1 month to about 50 years.

36. The method of claim 14, wherein the capsules can be administered at least once a day.

37. The method of claim 1, wherein a dose of the composition, is about 0.80 grams to about 1.50 grams.

38. The method of claim 1, wherein the individual is a human individual.

39. A powder composition for use in an individual, wherein the powder composition comprises glucomannan, xanthan gum, magnesium metal powder, an organic acid, excipients, or any combination thereof.

40. The powder composition of claim 39, wherein the powder composition is (i) contacted with water to form a hydrogel, and (ii) co-administered with food into the alimentary canal of an individual.

41. The powder composition of claim 39, for use in inducing satiety/weight loss in an individual.

42. The powder composition of claim 39, for use in inducing exercise endurance in an individual.

43. The powder composition of claim 39, for use in inducing anti-inflammatory effects in an individual.

44. The powder composition of claim 39, wherein a single dose of the powder composition comprises from about 3.0 grams to about 10.0 grams of glucomannan (GMN), xanthan gum (XG), magnesium metal powder (MMP), or any combination thereof.

45. The powder composition of claim 39, wherein the powder composition further comprises from about 30 wt % to about 60 wt % of GMN.

46. The powder composition of claim 39, wherein the powder composition further comprises from about 30 wt % to about 60 wt % of XG.

47. The powder composition of claim 39, wherein the powder composition further comprises from about 0.1 wt % to about 20 wt % of MMP.

48. The powder composition of claim 39, wherein the powder composition further comprises from about 0.01 wt % to about 25 wt % of an organic acid.

49. The powder composition of claim 39, wherein the powder composition further comprises from about 0.001 wt % to about 10 wt % of an excipient.

50. The powder composition of claim 39, wherein the composition comprises an organic acid such as citric acid, malic acid, succinic acid, tartaric acid, adipic acid, lactic acid, or any combination thereof.

51. The powder composition of claim 39, wherein the composition comprises excipients such as sweeteners, antioxidants, anticaking agents, flavoring agents, coloring agents, or any combination thereof.

52. The powder composition of claim 39, wherein the composition comprises sweeteners such as sucralose, stevia, sugar alcohol (e.g., erythritol), acesulfame, sucrose, glucose, fructose, aspartame, saccharin, cyclamate, agarose, or any combination thereof.

53. The powder composition of claim 39, wherein the composition comprises antioxidants such as ascorbic acid, isoascorbic acid, vitamin E, polyphenols, or any combination thereof.

54. The powder composition of claim 39, wherein the composition comprises anticaking agents such as tricalcium phosphate, powdered cellulose, magnesium stearate, sodium bicarbonate, sodium silicate, stearic acid, or any combination thereof.

55. The powder composition of claim 39, wherein the composition comprises flavoring agents such as lemon, chocolate, cherry, banana, pineapple, grape, wintergreen, or any combination thereof.

56. The powder composition of claim 39, wherein the composition comprises coloring agents such as riboflavin, carmel, annatto, chlorella, turmeric, elderberry, or any combination thereof.

57. The powder composition of claim 39, wherein, following co-administration the hydrogel expands by a volume of about 80 times to about 200 times the volume originally occupied by the powder composition contacted with water.

58. The powder composition of claim 39, wherein, prior to co-administration, the powder composition expands to form a hydrogel, comprising a volume equivalent to about 15% to about 60% of the individual's stomach capacity.

59. The powder composition of claim 39, wherein up to four doses of the powder composition in hydrogel form comprises a volume of about 8 ounces to about 16 ounces.

60. The powder composition of claim 40, wherein the hydrogel comprises a viscosity of at least about 15,000 millipascal-second at a temperature of about 72 degrees Fahrenheit.

61. The powder composition of claim 40, wherein the hydrogel comprises a volume sufficient to induce satiety in the individual.

62. The powder composition of claim 40, wherein the hydrogel comprises a volume sufficient to induce weight loss in the individual.

63. The powder composition of claim 40, wherein the hydrogel remains at a constant volume within the stomach of the individual for at least about 3 hours to at least about 8 hours.

64. The powder composition of claim 39, wherein the powder composition is packaged as a powder.

65. The powder composition of claim 39, wherein the powder composition is packaged in about 30 dose to about 60 dose packets.

66. The powder composition of claim 39, wherein the powder composition is packaged in about 5 gram to about 10 gram single dose packets.

67. The powder composition of claim 39, wherein the powder composition is enclosed in a capsule to form encapsulated composition.

68. The encapsulated composition of claim 67, wherein the encapsulated composition is enclosed in the capsule that is at least 0, 00, or 000 in size.

69. The encapsulated composition of claim 67, wherein the encapsulated composition is enclosed in the capsule comprising cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate, hydroxypropyl methylcellulose (HPMC), gelatin, polysaccharide, or any combination thereof.

70. The encapsulated composition of claim 67, wherein the encapsulated composition is enclosed in the capsule comprising hydroxypropyl methylcellulose (HPMC), cellulose, gelatin, or any combination.

71. The encapsulated composition of claim 67, wherein the encapsulated composition is released from the capsule following co-administration.

72. The powder composition and/or hydrogel from any one of claims 39 to 71, wherein the powder composition and/or hydrogel can be co-administered at least once a day.

73. The powder composition, encapsulated composition, and/or hydrogel from any one of claims 39 to 71, wherein the powder composition, encapsulated composition, and/or hydrogel can be co-administered at least once a day.

### EXAMPLES

The following illustrative examples are representative of embodiments of compositions and methods described herein and are not meant to be limiting in any way.

### Exemplary Embodiments

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. Among the exemplary embodiments are:

### XII. Examples

The following examples are included for illustrative purposes only and are not intended to limit the scope of the invention.

### Example 1: In Vitro Study of Delivery of 000 Vegetable Containing GMN/XG to an Aqueous Acidic Solution

### Introduction

This study was carried out to determine the potential of a 50:50 Glucomannan (GMN):Xanthan Gum (XG) mixture, when delivered to an acidic solution from an 000-vegetarian capsule, to gel 300 mL of the mixture. It is noted than magnesium metal powder (MMP) is not present in this formulation.

### Materials, Methods and Procedures

Material used here include: Konjac Glucomannan (GMN, manufactured by Hubei), Xanthan Gum (XG—manufactured by Fufung), distilled white vinegar (5% acetic acid, sold by Sam's West, Inc., or Great Value, sold at Walmart), 000 vegetable capsules (Pullulan, Purecaps, USA). Three 000 capsules were filled with a 50:50 mixture of GMN:XG. The gross weight of the three capsules was 2.9 grams and the net weight was 2.4 grams. The three full 000 capsules were immersed in 300 mL of white vinegar in a 350 mL plastic cup. The mixture was agitated for 30 seconds with a milk frother, at 10-minute intervals, for 60-minutes. Volume expansion was measured by marking the cup at the liquid-gel interface—before and after standing for 60-minutes. Viscosity measurements, on the mixture, were made with a Brookfield type viscometer (12 speed, #4 spindle) after standing for 60-minutes. The 300 mL mixture was transferred to a 1,000 mL beaker containing 700 mL of ROW (reverse osmosis water) for a total of 1,000 mL volume. A photo of the resultant solution in a beaker was taken (FIG. 1).

### Results

There was a lack of volume expansion as a result mixing and dissolution of three 000 capsules containing GMN:XG in 300-mL vinegar—and allowing for 120 minutes of dissolution. The resultant viscosity of the 300 mL mixture was 3,500 mPa·s. When mixing this 300 mL acidic solution with water (ROW), a phase separation takes place, where a substantial portion of the 300-mL mixture sinks to the bottom of the 1,000 mL beaker while the remaining portion, containing the non-dissolved capsular material—floats. Two photos of the resultant mixture were taken. FIG. 1 contains a horizontal view, while FIG. 2 contains a vertical view.

### Discussion of the Results in FIG. 1 and FIG. 2

There was a lack of volume expansion due to mixing GMN:XG with acidic water, as well as a phase separation in excess solution. Testing for volume expansion of the GMN:XG mixture was carried out—for comparison with mixtures of GMP:XG:MMP (magnesium metal powder)—as will be discussed in the subsequent Examples. That is, this experiment shows the lack of volume expansion of a GMN:XG mixture. Previous results (unpublished) have indicated that neither GMN, XG, or mixtures of such, undergo significant volume expansion on standing in aqueous solutions.

The viscosity of the 300 mL solution, i.e., 3,500 mPa·s—is comparable to the viscosity of 100% glycerol, i.e., 4,000 mPa·s. Thus, it can be concluded that a mixture of aqueous GMN:XP forms a weak hydrogel or a viscous solution.

Observing FIGS. 1 and 2, it can be seen that a phase separation takes place when the 300 mL of the acidic GMN:XG viscous solution is added to the 700 mL of ROW (reverse osmosis water), it can be concluded that a significant portion of the GMN:MMP capsule material does not properly dissolve in 5% aqueous acetic acid within the 120-minute period of this Experiment. That is, all material that has become saturated with the 5% aqueous acetic acid solution should ‘sink’—since 5% aqueous acetic acid is denser than water. Therefore, the capsular material observed floating has not been in contact with the acidic solution.

Previously, it has been observed that capsules containing glucomannan, alone, retain their capsular shape and do not expand significantly in volume—due to their remaining encapsulated—and not gaining full access to the aqueous solution.

In summary, GMN, XG or mixtures of such, when delivered from capsules, do not have the capability of gelling 300 mL of an acidic solution or affecting its volume expansion—due to the GMN and XG material remaining trapped in the capsule.

It should be noted that the behavior of GMN/XG powder contained in capsules is different than when GMN/XG is dispersed into a solution—as a non-encapsulated powder. When the latter is dispersed as a fee flowing powder into water at 0.7% w/v, or higher concentration of GMN/XG, and mixed, the solution forms a highly viscosity gel.

### Example 2: Gelling of Encapsulated GMN:XG:MMP (Magnesium Metal Powder):Citric Acid (CA) Powders Delivered to 5% Aqueous Acetic Acid—from 000 Vegetable Capsules

### Introduction

A study was carried out to determine the gelling capacity of eight different formulations of GMN, XG, MMP and CA as delivered to 5% aqueous acidic solutions. The formulations studied are listed in Table 1. This Study was carried out to test for gelling and volume expansion as well as to determine the factors that are important in affecting volume expansion.

### Materials and Methods

Formulations 1-8 of Table 1 (Columns 3-6) were prepared by mixing the ingredients and filling 000 cellulose capsules with the compositions. One capsule was immersed in 100 mL of white vinegar in a 7-ounce plastic cup. The mixtures were agitated for 30 seconds with a milk frother—at 10-minute intervals, for 60-minutes. Volume expansion was measured by marking the cup at the liquid-gel interface-before and after standing for 60-minutes. A gravimetric method was used to measure volume expansion (i.e., obtaining the weight of solution in the cup for before and for after volume expansion). Viscosity measurements, on the mixtures, were made with a rotary—type viscometer (12 speed, #4 spindle) after standing for 60-minutes at about 72 deg. F.

### Results

The results in Column 2 of Table 1 show the hydrogel volume created due to the presence of aqueous-acidic solutions of glucomannan (Column 3), magnesium metal powder (Column 4), citric acid (Column 5) and xanthan gum (Column 6). Of significance, is 167.8 mL hydrogel created by the composition of Formula #8 containing both GMN and XG along with MMP.

TABLE 1
Volume Gelled in Aqueous Acid
Formulations of & GMN/XG MMP/CA

| | | | | | | | GMN/ | |
|---|---|---|---|---|---|---|---|---|
| | Gel | Capsule | GMN | MMP | CA | XG | XG | % Effi- |
| | Vol. | Num- | mg./ | mg./ | mg./ | mg./ | % Effi- | ciency/ |
| ID | mL | ber(000) | cap | cap | cap | cap | ciency | mg. MMP |
| 1 | 81.4 | 1 | 778 | 111 | 37 | 0 | 105 | 1.0 |
| 2 | 82.3 | 1 | 824 | 55 | 23 | 0 | 100 | 1.8 |
| 3 | 68.3 | 1 | 857 | 37 | 37 | 0 | 79.7 | 2.2 |
| 4 | 50.8 | 1 | 895 | 38 | 0 | 0 | 57 | 1.5 |
| 5 | 62.7 | 1 | 922 | 19 | 0 | 0 | 68 | 3.6 |
| 6 | 154.6 | 1 | 761 | 160 | 80 | 0 | 203 | 1.3 |
| 7 | 119.8 | 1 | 828 | 78 | 39 | 0 | 145 | 1.9 |
| 8 | 167.8 | 1 | 398 | 167 | 84 | 398 | 211 | 1.3 |

Abbreviations: GMN—glucomannan; MMP—magnesium metal powder; CA—anhydrous citric acid; XG—xanthan gum

### Discussion

The goal, here, is to develop a formulation that will gel 300 mL, or more, of acidic stomach contents. Ingesting the encapsulated ingredients that gel 300+mL of acidic stomach fluid would limit the amount of food that can be ingested. Using the information derived from the formulations tested here, we can estimate the composition of ingredients and number of capsules necessary to accomplish this goal. For regulatory purposes, we need to limit the amount of MMP to 80 mg. per three-000 capsules or 26.7 mg. per cap for a three-capsule dose. The most efficient dose in forming a hydrogel with GMN, comes about when it is combined with XG. Therefore, to attain the goal of gelling the aqueous acidic solution most efficiently, GMN:XG:MMP formulations were subject to further study. It was determined that citric acid was not necessary to hydrogel development—since stomach acid provides the catalyst for reaction of MMP with water.

### Example 3: Study of GMN:XG:MMP Formulations in Aqueous Acidic Solutions

### Introduction

From analyzing the results presented in Table 1, it is clear that gelling 300 mL of an acidic solution, which using GMN, alone with MMP, would not be sufficient to gel 300 mL of aqueous acidic solution using only three-000 capsules. The results presented in Table 1 above, indicate that combining GMN with XG in the presence of MMP increased the efficiency of gel formation and expansion. Thus GMN:XG:MMP combinations become the subject of additional studies as described below.

### Methods and Materials

Columns 2-4 of Table 2 display the compositions of Formulas 1S to 4S that were the subject of testing described in this Example. After mixing the ingredients and filling into 000 cellulose capsules (Column 5 of Table 2), four—capsules of each formulation were independently immersed into 300 mL of 5% acetic acid. Then, the resultant suspensions we mixed for 30-seconds at 10-minute intervals for a total of 60-minutes. Volume expansion was measured by marking the cup at the liquid and gel interface—after standing for 60-minutes. A gravimetric method was used to measure volume expansion. Viscosity measurements, on the mixtures, were performed with a rotary type viscometer (12 speed, #4 spindle)—after standing for 60-minutes.

### Results

Table 2 displays the resultant hydrogel volumes (Column 7, Table 2) and viscosities (Column 8, Table 2) of the three encapsulated GMN:XP:MMP formulations (2S-4S)—relative to an encapsulated GMN:MMP control (1S). The 1S encapsulated GMN:MMP control formulation gels 288.9 mL of acidic solution, while the three GMN:XG:MMP formulations (2S-4S) gel 318.5-356.9 mL of acidic solution (Column 6, Table 2). All three GMN/XG/MMP encapsulated formulations, when interacting with the acidic solution, generate viscosities of between 13,000 and 25,500 mPa·s—resulting from the gelation and expansion 300 mL of acidic solution.

It is worth noting that the encapsulated GMN/MMP formulation (1S of Table 2), when mixed with 300 mL of 5% acetic acid affects a reduced hydrogel volume of 288.9 mL—but has a higher viscosity i.e., 31,500 mPa·s—than the other encapsulated formulations, indicating it forms a more compact hydrogel. This phenomenon is unexpected, since, as noted in USA PA 16736749, when free, non-encapsulated GMN/MMP formulations are mixed with water, hydrogel volume expansion of 10-70% takes place.

TABLE 2
Gelation and Viscosity Study GMN/XG/MMP Solutions

| | gm./ | gm./ | gm./ | | | Gel | |
|---|---|---|---|---|---|---|---|
| | cap | cap | cap | gm./Cap | No. of | Volume | Viscosity |
| ID | GMN | XG | MMP | Net Wt. | Capsules | mL | mPa · s |
| 1S | 0.93 | 0 | 0.02 | 0.833 | 4 | 288.9 | 31,500 |
| 2S | 0.47 | 0.47 | 0.02 | 0.876 | 4 | 356.9 | 25,500 |
| 3S | 0.62 | 0.31 | 0.02 | 0.862 | 4 | 328.6 | 13,000 |
| 4S | 0.75 | 0.19 | 0.02 | 0.876 | 4 | 318.5 | 23,000 |

Abbreviations: GMN—glucomannan; MMP—magnesium metal powder; CA—anhydrous citric acid; XG—xanthan gum; gm—grams; cap—000 capsule; wt.—weight; mL—mililiters; mPa · s—millipascal per second

### Discussion

From an analysis of the results displayed in Table 2, it can said that it has been discovered that a combination of encapsulated GMN and XG, containing MMP, is a more efficient and effective entity at gelling 300 mL of an acidic solution as compared to GMN with MMP. In fact, the encapsulated GMN:MMP formulation gels only 288.9 mL of the 300 mL solution-indicating hydro gel volume shrinkage—a unexpected result.

### Example 4: Effect of Three and Four ‘000’ Capsules, Containing GMN:XG:MMP, on Gelation, Viscosity and Floatation of Resultant Hydrogels

### Introduction

Although use of four-000 capsules containing GMN:XP:MMP formulations have been shown to be more than sufficient to gel 300 mL of acidic solution (See Table 2), it is preferable from an ease of consumption point of view, to be able to gel the acidic solution with the contents of the lowest number of capsules. In an attempt to accomplish this objective, the concentration of MMP, as well as the other ingredients in the formula were adjusted to a total dose of 80 mg. MMP for the capsules tested.

### Materials and Methods

In this Study, the formulation 1S, (0.46 g. GMN; 0.46 g. XG; 0.27 mg/capsule) of the previous Example, is designated a 1T when testing 3 capsules and as 4T when testing 4-capsules. Formulas 2T and 3T were prepared in 000 cellulose capsules and are displayed in Columns 2-4 of Table 3.

Three or four (i.e., four for T4)—capsules of each formulation were each immersed in different 300 mL volumes of 5% acetic acid. Then, the resultant mixtures we mixed for 30-seconds at 10-minute intervals for a total of 60-minutes. Volume expansion was measured by marking the cup at the liquid-gel interface after standing for 60-minutes. A gravimetric method was used to measure volume expansion. Viscosity measurements, on the mixtures, were made with a rotary type viscometer (12 speed, #4 spindle) after standing for 60-minutes. Floatation of material was visually observed and photographed.

### Results

### Discussion

Observing the resultant hydrogel volumes after incubation of 3-capsules of 1T and 2T, it is observed that 3-capsules are sufficient to not only gel 300 mL of 5% acetic acid solution—but to expand its volume to 349-372 mL. Also, of interest, is the observation that using 3-capsules as compared to using 4-capsules (Compare 1T to 2T) affects a decrease in viscosity and an increase in hydrogel volume of the 5% acetic acid solution. This observation demonstrates a ‘tighter’ hydrogel structure when gelling the acidic aqueous solution with the contents of four capsules compared to using 3-capsules. All of the GMN:XG:MMP hydrogels tested, as listed in Table 3, generated a hydrogel of more than 300 mL of 5% acidic solution.

### Example 5: Effect of Mixing a GMN:XG:MMP Aqueous Acidic Hydrogel with Simulated ‘Chyme’

### Introduction

The protocol for ingestion of the encapsulated formulation entails taking three capsules with 10-12 ounces (around 300 mL) of water—‘just’ before or during a meal. Taking the capsules, with water, just before or during a meal is done to generate a hydrogel that will occupy about 25-30% of the stomach with a volume of fluid so that consumers ‘feel full’—thereby eating less food. However, the stomach will ‘chum’ the bolus resulting in the mixing of the capsule contents with the meal. Thus, testing the resultant hydrogel properties with a simulated ‘chyme’ (stomach contents) should be informative.

### Materials, Methods and Results

Three 1T capsules (see Table 3) were added to 300 mL of 5% acetic acid and mixed with a milk frother for 30-seconds, six times over—a 60-minute period. The final hydrogel volume expanded to about 350 mL (See the cup on the left side of FIG. 4). The viscosity, at 72 F, was 4,250 mPa·s.

A simulated food sample was prepared consisting of corn starch (30 g.), fish collagen (25 g.), citric acid (10 g.), MCT powder (15.0 g.), and egg yolk protein (20.0 g.) for a total of 99.7 grams. The simulated food sample was mixed with 650 mL of 5% aqueous acetic acid—using a kitchen utensil. A yellow, turbid suspension was formed (See the right side of FIG. 4). The viscosity of this combination of food and 5% aqueous acetic acid mixture, at 72 F, was 3.5 mPa·s.

Mixtures 1 and 2, above, were combined, observations and viscosities performed:
1. Before mixing. ‘1’ was seen ‘floating’ on top of ‘2’, (see FIG. 5).
2. After vigorous mixing of ‘1’ with ‘2’, and allowing it to stand, the mixture settled into three Phases (see FIG. 6).
3. The viscosities of the 3-phases were: Top Phase: 383 mPa·s; Mid Phase: 5 mPa·s; Bottom Phase: 3 mPa·s—at 72 about degrees Fahrenheit (F).

### Discussion

During the process of digestion, the stomach, through peristaltic action and contractions ‘chums’ ingested food into a ‘chyme’ that has been mixed with stomach acid and digestive enzymes. The in vitro results of this Study indicate that when mixing the 1T mixture with the simulated food mixture—that the 1T mixture will rise to the top of the vessel—as seen in the photos displayed in FIGS. 4 and 5. Thus, the viscous mixture, generated from 1T, will tend to reside longer in the stomach—which would affect satiety. Mixing the 1T material with the simulated food, reduces the viscosity of the ‘floating’ hydrogel. Mixing food with a gelling material is be expected to do so. This mixing process should slow down the digestion of food—since interference in enzyme-substrate interactions is likely to take place.

### Example 6: Time Course of Residence of a GNM:XG:MMP Hydrogel Bolus in the Stomach

### Introduction

Given that a 300 mL hydrogel affects satiety when occupying 25-30% of the stomach volume, it would be of interest to determine how long it will reside in the stomach thereby prolonging satiety. A long-lasting product is more likely to reduce hunger and affect weight loss. A 1984 US patent (U.S. Pat. No. 4,548,805) teaches that “A non-invasive method for determining gastric acidity in humans, using oral magnesium metal powder while measuring the hydrogen gas response in exhaled breath and belches. The magnesium reacts with water, in the presence of hydrochloric acid, thereby liberating hydrogen and the magnitude of the hydrogen released is correlated with the amount of gastric acid produced.” Since our formulation contains magnesium metal powder (MMP), we can employ this method, using H2 as a tracer, to determine the residence time of the formulation, to be tested, in the stomach. That is, MMP reacts with water in the presence of stomach hydrochloric acid. When MMP, from the formulation, passes form the highly acidic environment of the stomach—to the more alkaline small intestine, the pH abruptly rises and the reaction of MMP with water is reduced—signaling that the formulation contents have passed to the small intestine—from the stomach.

### Materials and Methods

The time course of generation and sustainability, in the stomach, of the orally administered, encapsulated 1T formulation—was determined by the measurement of breath hydrogen (H2). For breath H2 measurements, a modified Forensics Detectors® H2 testing instrument—was used. It was acquired along with a Forensics Detectors® Breath H2 testing Kit, including a desiccant trap for protection of the instrument electrodes from excessive moisture. For each measurement, a deep breath was taken, held for 3-seconds and the breath was expelled through a tube—into the portal of the instrument—for about 20 seconds—until a light, on the instrument, ‘shut off’—indicating for the user to stop expelling the breath into the instrument. A healthy adult male was used as the subject.

For this Study, the subject fasted for 10-hours-overnight. A baseline measurement of breath H2 was taken before ingestion of any substances. Thereafter, three capsules of Formula 1T (GMN:XG:MMP), delineated in Table 3, were taken with 300 mL of water—5-minutes before ingestion of a meal. The meal consisted of a turkey patty, whole wheat bread sandwich, a 600 mL ‘smoothie’ and nutritional supplements plus the 300 mL of water.

### Results

FIG. 7 displays the time course of generation and sustainability of breath H2 in the stomach. The shape of the time course plot is complex due to the interactions that take place during digestion—including interaction of stomach acid and food with the capsules, partial neutralization of acid by food components and the effects of stomach contractions, peristalsis and periodic secretions of aqueous acid interactions that effect the reaction of magnesium metal with water.

It should be noted that the subject experienced a sensation of fullness for four hours after ingesting the capsules—until being served his next meal.

### Discussion

Of importance is the observation that the formulation of interest, in the presence of food, has the potential to be retained in the stomach for up to 8 hours. In turn, it may affect satiety for close to that time.

### Example 7: Dose-Response of Magnesium Metal Powder (MMP) on Gelling and Volume Expansion Due to GMN:XG:MMP Capsules

### Introduction

Having demonstrated that MMP is essential for gelling and volume expansion of GMN:XG:MMP—as delivered from capsules, it is of interest to investigate the effect of the concentration of MMP on these parameters.

### Materials and Methods

000 cellulose capsules filled with 1:1.2 ratio of GMN:XG:—containing different doses of MMP, were studied (see the formulations listed in Table 4). One capsule containing a specified formulation was added to 100 mL of vinegar and mixed (frothed) for 30-seconds, at 10-minute intervals for 60 minutes. Observations were made and viscosity measurements taken—after allowing the mixtures to settle for 2-hours.

### Results

Observations during the time course of the Study suggest that all test objects were building viscosity and undergoing some volume expansion. At 2-hours after the beginning of the Study, viscosity and volume expansion measurements were made. Viscosities ranged from 13,000-15,000 mPa·s—while the excess volume expansions (Column 7 of Table 4) ranged from 3.5-19.1 mL, 72 degrees Fahrenheit (F).

TABLE 4
Dose-Response MMP Effect of Gelling
of GMN:XG 1:1.2 in 5% Acetic Acid

| | | | Viscosity | Vol. Expansion |
|---|---|---|---|---|
| | Ingredient | Grams | mPa · s | mL |
| 1 U | Glucomannan | 10.63 | 13,750 | 8.3 |
| | Xanthan Gum | 12.63 | | |
| | MMP | 0.667 | | |
| | Total | 23.927 | | |
| | Net wt. of matrrial in a cap: 0.876 g. | | | |
| 2 U | Glucomannan | 10.63 | 15000 | 3.5 |
| | Xanthan Gum | 12.63 | | |
| | MMP | 0.500 | | |
| | Total | 23.76 | | |
| | Net wt. of matrrial in a cap: 0.909 g. | | | |
| 3 U | Glucomannan | 10.63 | 13,000 | 8.3 |
| | Xanthan Gum | 12.63 | | |
| | MMP | 0.834 | | |
| | Total | 24.094 | | |
| | Net wt. of matrrial in caps: 0.895 g. | | | |
| 4 U | Glucomannan | 10.63 | 13,750 | 19.1 |
| | Xanthan Gum | 12.63 | | |
| | MMP | 1.043 | | |
| | Total | 24.303 | | |

Net wt. of matrrial in caps: 0.894 g.

### Discussion

All four capsular formulations tested were able to build significant viscosities of their mixtures—with 5% acetic acid. More diverse, was their volume expansions, where the GMN/XG/MMP formulation containing 4.4% MMP had the largest volume expansion.

### Example 8: Gel Formation from 3-Capsules of Formulations 1U and 4U (Table 4) in a 400 mL of 5% Acetic Acid

### Introduction

Since the stomach may contain more than 300 mL of acidic water, an experiment was carried out to determine the gelling effect of 3-capsules of formulas 1U and 4U (Table 4).

### Method

Three capsules of 1U and 4U, respectively were added to two 400 mL volumes of 5% aqueous acetic acid in 500 mL beakers and then mixed, as described above, and allowed to ‘stand’ for an hour—after mixing. The viscosities and volume expansions were measured.

### Results

At 1-hour post-mixing, a photo was taken and is displayed as FIG. 8. It can be seen that both capsular formulations affect gelling. Not only do the 400 mL volumes gel, but a volume expansion also rising to 510-535 mL is observed to have taken place. Measurement of the viscosities of the gel associated with 1U yielded 10,000 mPa·s while that associated with 4U yielded 9,000 mPa·s.

The volume expansion associated with both 1U and 4U holds—overnight (10-12 hours) even though the gels were left standing—uncovered overnight at 72 F. The viscosities increased to 49,500 for 1U and to a lesser extent of 11,750 for 4U (FIG. 9).

### Discussion

The results show the remarkable ability of GMN:XG:MMP formulations as delivered from three—000 capsules, to not only gel 457 times their weight of acidic water (volume, as well), but to expand the 400 mL hydrogel volume to about 525 mL—a 31% increase in hydrogel volume—above the original volume occupied by the acidic solution. If a consumer took three capsules of 1U with 13.5 ounces of water, she has the potential to have a hydrogel occupy 525 mL of her stomach, or about 52.5% of an average sized stomach.

### Example 9: Gel Formation from 2-Capsules of Formulation 1U (Table 4) in a 400 mL Volume of 5% Acetic Acid

### Introduction

After observing the remarkable effect that three-000 capsules containing, 1U, has on gelling and expanding the volume of 400 mL of 5% aqueous acetic acid, it became of interest to determine the effect of two capsules of 1U on the gelling and expanding of 400 mL of 5% aqueous acetic acid.

The rationale for testing two capsules of 1U is that some people are refractory to swallowing a larger number of 000 capsules.

### Method

Two capsules of 1U (Table 4) were added to 400 mL vinegar, mixed, and observed, as described above, and allowed to ‘stand’ for an hour—after mixing. The viscosities and volume expansions were measured.

### Results

At 1-hour after mixing, a bubbly gel was formed that occupied a volume of about 425 mL. The viscosity of this ‘gel’ was determined to be 8,750 mPa·s. After standing—overnight (72 F), the viscosity held at 7,250 mPa·s. To further determine the integrity of this gel, it was transferred to 1,000 mL beaker containing 400 mL of 5% aqueous acetic acid. As can be observed in FIG. 10, an approximately 400 mL of gel floats on 5% aqueous acetic acid and holds its integrity—without intensive mixing.

Thereafter, the total 850 mL volume was intensively mixed for 60-seconds with a milk frother. The mixture was allowed to stand for about 2.5 hours. A uniform, turbid viscous solution of a viscosity of 255 mPa·s was formed.

### Discussion

The results show the remarkable ability of GMN:XG:MMP formulations, delivered from as few as two-‘000 capsules, to form an 8,500 mPa·s gel—that floats on the 5% acidic acid solution. Even when this gel is disrupted by rigorous mixing, with twice its volume, it forms a thixotropic solution of viscosity 255 mPa·s that could slow down the digestion of food in the G-I tract—after it passes through the stomach—to the small intestine.

### Example 10: Effect on Satiety—of Ingestion of 3-Capsules Containing 1U

### Introduction

The results of the above studies suggest that ingestion of 3-capsules of an effective gelling and expanding formulation of GMN:XG:MMP, e.g., 1U, should affect satiety. With this thought in mind, the following study was carried out.

### Materials and Methods

The adult, male subject fasted—overnight for approximately 12-hours. Baseline breath hydrogen (H2) measurements were taken before dosing and eating. Ten minutes before his 9:00 AM meal, he consumed 3-capsules of 1U (see Table 4) with about 16-oz. of reverse osmosis water. The lunch meal consisted of a turkey patty sandwich, a nutritious ‘smoothie’ and nutritional supplements. Breath H2 was measured periodically until completion to the Study—when the evening meal was consumed at 6:00 PM.

### Results

After completion of the 9:00 AM meal, the subject went about his duties and felt satiated up to being served his 5:30 PM meal. There were no side effects experienced such as bloating or belching. He was able to eat all of his evening meal but avoided desert.

Breath hydrogen measurements indicated that the gel formed by 3-capsules could have remained in the stomach for up to 7-hours.

### Discussion

Two of the major reasons that individuals put on excess weight—or cannot lose excess body weight is ‘snacking’ in-between meals and over-eating at meals.

The GMN:XG:MMP formulations tested here, promise to reduce food intake, and affect satiety so that significant weight loss or weight control can take place.

### Example 11: Comparison of Viscosities and Hydrogel Volumes Generated by Glucomannan:Xanthan Gum:Magnesium Metal Powder in Capsules and in a Powder Formulation when Mixed with 5% Acetic Acid

### Introduction

Previously, it has been shown (U.S. Ser. No. 16/736,749—pending) that glucomannan (GMN) can sequester molecular hydrogen—generated by the reaction of magnesium metal powder (MMP)—with water. This sequestration leads to a marked volume expansion of the aqueous acidic solution harboring the GMN-MMP complex—and forming a hydrogel of high viscosity of the order of 25,000 mPa·s. The same phenomenon takes place when xanthan gum (XG) is added to this complex over a wide range of ratios of the concentrations.

Furthermore, it has been discovered that when GMN/XG/MMP complexes are encapsulated and incubated in aqueous acid solution—that their dispersion, into solution and gelling potential—is reduced. As a result, a comparison study of viscosity of GMN/XG/MMP hydrogels resulting from both capsules and a powder formulation has been carried out.

### Methods and Materials

Different dosage forms were tested:
1. GMN(44.4%)/XG(52.8%)/MMP(2.8%) Intact 00 HPMC Capsules into acidic solution.
2. GMN(44.4%)/XG(52.8%)/MMP(2.8%) Intact 000 HPMC Capsules into acidic solution.
3. H2-HydroGel powder containing 22.2% combination of GMN/XG/MMP plus 77.8% excipients.
4. Each 00 HPMC capsule contains 0.635 g. net weight of powder material. Each 000 HPMC capsule contains a net weight of 0.962 grams of powder. The H2-Hydrogel powder contained a total of 6.6 grams.

For each dosage form, a defined number of intact capsules a dose of the Hydrogel powder was added to 400 mL of 5% acidic solution. An electric mixer, adjusted to a stirring speed of 1,000 rpm, was allowed to stir the solution for one—hour. After standing for 30-minutes, the viscosities of the solution/gel were measured. Viscosities were measured with a rotary style viscometer. Rotor speed and spindles were interchanged according to the viscosity of the solution/gel. Temperature (72 degrees Fahrenheit) and the molecular hydrogen content of the solution were measured after viscosity measurements were taken.

### Results and Discussion

The results of the viscosity measurements are displayed in Table 5 and plotted in FIG. 11.

TABLE 5
Compare Viscosities of Different Dosage forms: 000 HPMC
Capsules, 00 HPMC Capsules and H2-HydroGel from Powder
Viscosity mPa · s

| | GMN/XG/MMP | GMN/XG/MMP | GMN/XG/MMP+ |
|---|---|---|---|
| | 00 HPMC | 000 HPMC | H2-HydroGel |
| # | Capsules | Capsules | from Powder |
| Units | Capsules | Capsules | Scoops |
| 0 | 0 | 0 | 0 |
| 1 | 62 | 375 | 1,000 |
| 2 | 1440 | 3,100 | 37,000 |
| 3 | 3,700 | 6,100 | 80,000 |
| 4 | 5,000 | 13,500 | 99,000 |
| 5 | 14,000 | 26,000 | |
| 6 | 26,500 | 37,500 | |
| 7 | 34,500 | 99,000 | |
| 8 | 99,000 | | |
| 9 | 99,000 | | |

Comparing the results displayed in Table 5 and FIG. 11, the following observations are worthy of noting:
1. For all three viscosity vs. # of Units (capsules or dose of powder), the plots—display an exponential increase in viscosity—as the dose is increased. This exponential increase may be a result of self-interaction of the GMN/XG complex in an aqueous acidic solution.
2. Comparing curves for the GMN/XG/MMP capsules (red & blue curves) with that of the GMN/XP/MMP powder (grey), it is obvious that incapsulating GMN/XP/MMP in both 00 HPMC and 000 HPMC capsules retards the viscosity increase when the dose is less than 6-7 capsules. However, at a dose of 6-7-capsules, the viscosities generated by the equivalent amount of powder is equal to the viscosity generated by the capsules. To generate a strong viscosity of around 37,000 mPa·s, in 400 mL of acidic solution, it takes two doses of the H2-Hydrogel powder, six capsules of the 000 HPMC capsules and seven capsules of the 00 HPMC capsules.

Off hand, one could misinterpret the efficiencies of viscosity generation by the different dosage forms as shown in Table 5 and FIG. 11. That is, each dose (capsule or powder) contains a different amount of GMN/XG/MMP—which is the viscosity forming entity. That is, one 00 HPMC capsule contains 0.635 g. of GMN/XG/MMP; one 000 HPMC capsule contains 0.962 g. of GMN/XG/MMP, and one 6.6-gram scoop of the H2-HydroGel contains 1.48 grams of GMN/XG/MMP.

Extrapolating from the results of Table 5, for obtaining a viscosity of 37,000 mPa·s, it would take 2.8 grams of GMN/XG/MMP delivered from the H2-HydroGel; 5.8 grams of GMN/XG/MMP delivered from 000 GMN/XG/MMP capsules and about 4.8 grams of GMN/XG/MMP delivered from the 00 HPMC capsules. Thus, delivery of GMN/XP/MMP from the H2-HydroGel is more efficient in generating a robust viscosity compared to delivery from the said capsules.

At completion of the stirring protocol, H2 was detected (Trustlex H2-Meter) in solution/gels for the dosage forms tested, above, with exception of testing with just one or two capsules and for a single dose of the H2-HydroGel. It is likely that H2 escaped from the 400-mL beaker during the one hour of vigorous mixing due to the low concentration of GMN in those solutions or low viscosity gels.

Volume expansions, under the rigorous mixing conditions of the tests described above were low, i.e., 0-50 mL for the 00 HPMC GMN/XG/MMP capsules tested.

The viscosity results presented here, favor use of the H2-HydroGel as the most efficient delivery system. However, it should be kept in mind that the H2-HydroGel powder, itself, is not ingested, as such. Rather, the H2-SatietyGel is mixed with water and allowed to build viscosity and expand—before ingestion.

### Advantages of Consuming the H2-Hydrogel—Over Ingestion of the Capsules:

There is no risk of a gel forming in the throat or capsules getting stuck in the throat and developing a ‘gel’ that could cause choking.
By allowing MMP, in the H2-Hydrogel, to react in vitro before ingestion, the patient will not being ingesting MMP—they will be ingesting magnesium hydroxide.
The results presented in Table 5 would infer that as little as 2-scoops of the H2-Hydrogel would generate a viscosity of 37,000 cps. An effective weight loss dose could be two scoops.
Previously, in vitro, it has been that the H2-HydroGel, in an aqueous solution, can expand by 40-60% of its original liquid volume. Delivering GMN/XG/MMP from capsules does not display this large an expansion.

### Advantages of Consuming the Capsules—Over Consuming the H2-Hydrogel

Capsules are the traditional and expected means of consuming medication.
Capsules do not require mixing with water prior to ingestion.
GMP manufacturing of capsules is less complex than manufacturing of the H2-Hydrogel powder.
Capsules are more portable.
In contrast to consuming the H2-Hydrogel, capsules, taken with water-instantly. It takes more time to consume the H2-Hydrogel after it has been mixed with water.
   Regardless of dosage form, as described, above, providing an adequate dose that generates a viscosity of 37,000 mPa·s should be effective at inducing satiety. It is feasible to provide the patient with capsules during some meals and the H2-Hydrogel during other meals.

What is claimed is:

1. A method of administrating a composition capable of generating molecular hydrogen to an individual, the method comprising: co-administering food and a dose or doses of the composition to the alimentary canal of the individual, wherein the composition comprises glucomannan, xanthan gum, magnesium metal powder, or any combination thereof.

2. The method of claim 1, wherein the composition is enclosed in a capsule.

3. The method of claim 1, wherein the composition is formulated for oral delivery.

4. The method of claim 1, wherein the composition is formulated to induce satiety/weight loss.

5. The method of claim 1, wherein the composition is formulated to induce exercise endurance.

6. The method of claim 1, wherein the composition is formulated to induce anti-inflammatory effects.

7. The method of claim 1, wherein co-administration of food and a dose of the composition comprises releasing the composition into the stomach of the individual at most about 40 minutes following the introduction of food into the stomach of the individual.

8. The method of any one of claims 1 to 7, wherein the composition comprises at least about 2 wt % to at least about 98 wt % of glucomannan.

9. The method of any one of claims 1 to 7, wherein the composition comprises from about 40 wt % to about 50 wt % of glucomannan.

10. The method of any one of claims 1 to 7, wherein the composition comprises at least about 0.0 wt % to at least about 98 wt % of xanthan gum.

11. The method of any one of claims 1 to 7, wherein the composition comprises from about 40 wt % to about 50 wt % of xanthan gum.

12. The method of any one of claims 1 to 7, wherein the composition comprises at least about 0.001 wt % to at least about 30 wt % of magnesium metal powder.

13. The method of any one of claims 1 to 7, wherein the composition comprises from about 0.001 wt % to about 5 wt % of magnesium metal powder.

14. The method of any one of claims 1 to 13, wherein the composition is released from the capsule following co-administration.

15. The method of claim 1, wherein the composition is enclosed in the capsule that is at least 0, 00, or 000 in size.

16. The method of claim 1, wherein the capsule comprises cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate, hydroxypropyl methylcellulose (HPMC), gelatin, polysaccharide, or any combination thereof.

17. The method of claim 1, wherein the capsule is made from hydroxypropyl methylcellulose (HPMC), cellulose, gelatin, or any combination thereof.

18. The method of claim 1, wherein the method comprises co-administration of food and at least 2 capsules comprising a dose of the composition.

19. The method of claim 1, wherein the method comprises co-administration of food and at least 3 capsules comprising a dose of the composition.

20. The method of claim 1, wherein following co-administration of food and at least one capsule comprising a dose of the composition, the composition expands to a volume of about 100 milliliters to about 2,000 milliliters in acidic solution to form a hydrogel.

21. The method of claim 20, wherein the composition expands to a volume of about 300 milliliters.

22. The method of claim 1, wherein following co-administration of food and at least one capsule comprising a dose of the composition, the composition associates with acidic solution of the individual's stomach to form a hydrogel that expands to a volume of about 100 times to about 200 times the original volume of the capsule.

23. The method of claim 22, wherein the composition associates with acidic solution of the individual's stomach to form a hydrogel that expands to a volume of about 300 times the original volume of the capsule.

24. The method of claim 1, wherein following co-administration of food and at least one capsule comprising a dose of the composition, the hydrogel comprises a volume equivalent to about 15% to about 60% of the individual's stomach capacity.

25. The method of any one of claims 20 to 24, wherein the hydrogel comprises a volume sufficient to induce satiety in the individual.

26. The method of any one of claims 20 to 24, wherein the hydrogel comprises a volume sufficient to induce weight loss in the individual.

27. The method of any one of claims 20 to 24, wherein the hydrogel remains at a constant volume within the stomach of the individual for at least about 4 hours to at least about 7 hours.

28. The method of any one of claims 20 to 24, wherein the composition expands in acidic solution to form a hydrogel, generating molecular hydrogen by reaction of magnesium metal powder with the acidic solution.

29. The method of any one of claims 20 to 24, wherein the molecular hydrogen accelerates the dissolution of the capsule containing the composition in the acidic solution.

30. The method of any one of claims 20 to 24, wherein the molecular hydrogen induces satiety/weight loss, exercise endurance, anti-inflammatory effects, or any combination thereof.

31. The method of any one of claims 20 to 24, wherein the acidic solution is stomach acid.

32. The method of claim 30, wherein satiety or weight loss is induced following co-administration of food and at least one capsule comprising a dose of the composition when ingested at least once a day.

33. The method of claim 32, wherein daily co-administration of food and at least one capsule comprising a dose of the composition reduces body weight, induces exercise endurance, and/or induces anti-inflammatory effects in the individual over an extended period of time.

34. The method of claim 33, wherein an extended period of time comprises at least about 1 week.

35. The method of claim 33, wherein an extended period of time comprises about 1 month to about 50 years.

36. The method of claim 14, wherein the capsules can be administered at least once a day.

37. The method of claim 1, wherein a dose of the composition, is about 0.80 grams to about 1.50 grams.

38. The method of claim 1, wherein the individual is a human individual.

39. A powder composition for use in an individual, wherein the powder composition comprises glucomannan, xanthan gum, magnesium metal powder, an organic acid, excipients, or any combination thereof.

40. The powder composition of claim 39, wherein the powder composition is (i) contacted with water to form a hydrogel, and (ii) co-administered with food into the alimentary canal of an individual.

41. The powder composition of claim 39, for use in inducing satiety/weight loss in an individual.

42. The powder composition of claim 39, for use in inducing exercise endurance in an individual.

43. The powder composition of claim 39, for use in inducing anti-inflammatory effects in an individual.

44. The powder composition of claim 39, wherein a single dose of the powder composition comprises from about 3.0 grams to about 10.0 grams of glucomannan (GMN), xanthan gum (XG), magnesium metal powder (MMP), or any combination thereof.

45. The powder composition of claim 39, wherein the powder composition further comprises from about 30 wt % to about 60 wt % of GMN.

46. The powder composition of claim 39, wherein the powder composition further comprises from about 30 wt % to about 60 wt % of XG.

47. The powder composition of claim 39, wherein the powder composition further comprises from about 0.1 wt % to about 20 wt % of MMP.

48. The powder composition of claim 39, wherein the powder composition further comprises from about 0.01 wt % to about 25 wt % of an organic acid.

49. The powder composition of claim 39, wherein the powder composition further comprises from about 0.001 wt % to about 10 wt % of an excipient.

50. The powder composition of claim 39, wherein the composition comprises an organic acid such as citric acid, malic acid, succinic acid, tartaric acid, adipic acid, lactic acid, or any combination thereof.

51. The powder composition of claim 39, wherein the composition comprises excipients such as sweeteners, antioxidants, anticaking agents, flavoring agents, coloring agents, or any combination thereof.

52. The powder composition of claim 39, wherein the composition comprises sweeteners such as sucralose, stevia, sugar alcohol (e.g., erythritol), acesulfame, sucrose, glucose, fructose, aspartame, saccharin, cyclamate, agarose, or any combination thereof.

53. The powder composition of claim 39, wherein the composition comprises antioxidants such as ascorbic acid, isoascorbic acid, vitamin E, polyphenols, or any combination thereof.

54. The powder composition of claim 39, wherein the composition comprises anticaking agents such as tricalcium phosphate, powdered cellulose, magnesium stearate, sodium bicarbonate, sodium silicate, stearic acid, or any combination thereof.

55. The powder composition of claim 39, wherein the composition comprises flavoring agents such as lemon, chocolate, cherry, banana, pineapple, grape, wintergreen, or any combination thereof.

56. The powder composition of claim 39, wherein the composition comprises coloring agents such as riboflavin, carmel, annatto, chlorella, turmeric, elderberry, or any combination thereof.

57. The powder composition of claim 39, wherein, following co-administration the hydrogel expands by a volume of about 80 times to about 200 times the volume originally occupied by the powder composition contacted with water.

58. The powder composition of claim 39, wherein, prior to co-administration, the powder composition expands to form a hydrogel, comprising a volume equivalent to about 15% to about 60% of the individual's stomach capacity.

59. The powder composition of claim 39, wherein up to four doses of the powder composition in hydrogel form comprises a volume of about 8 ounces to about 16 ounces.

60. The powder composition of claim 40, wherein the hydrogel comprises a viscosity of at least about 15,000 millipascal-second at a temperature of about 72 degrees Fahrenheit.

61. The powder composition of claim 40, wherein the hydrogel comprises a volume sufficient to induce satiety in the individual.

62. The powder composition of claim 40, wherein the hydrogel comprises a volume sufficient to induce weight loss in the individual.

63. The powder composition of claim 40, wherein the hydrogel remains at a constant volume within the stomach of the individual for at least about 3 hours to at least about 8 hours.

64. The powder composition of claim 39, wherein the powder composition is packaged as a powder.

65. The powder composition of claim 39, wherein the powder composition is packaged in about 30 doses to about 60 dose packets.

66. The powder composition of claim 39, wherein the powder composition is packaged in about 5 grams to about 10 grams single dose packets.

67. The powder composition of claim 39, wherein the powder composition is enclosed in a capsule to form encapsulated composition.

68. The encapsulated composition of claim 67, wherein the encapsulated composition is enclosed in the capsule that is at least 0, 00, or 000 in size.

69. The encapsulated composition of claim 67, wherein the encapsulated composition is enclosed in the capsule comprising cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate, hydroxypropyl methylcellulose (HPMC), gelatin, polysaccharide, or any combination thereof.

70. The encapsulated composition of claim 67, wherein the encapsulated composition is enclosed in the capsule comprising hydroxypropyl methylcellulose (HPMC), cellulose, gelatin, or any combination.

71. The encapsulated composition of claim 67, wherein the encapsulated composition is released from the capsule following co-administration.

72. The powder composition and/or hydrogel from any one of claims 39 to 71, wherein the powder composition and/or hydrogel can be co-administered at least once a day.

73. The powder composition, encapsulated composition, and/or hydrogel from any one of claims 39 to 71, wherein the powder composition, encapsulated composition, and/or hydrogel can be co-administered at least once a day.
